SEQUENCE LISTING (17 sequences)

SEQ ID NO: 1 - DNA, 1602 bp, Homo sapiens
atgggtccgcgcggcgcggcgagcttgccccgaggccccggacctcggcggctgctcctccccgtcgtcctcccgctgctgctgctgctgttgttggcgccgccgggctcgggcgccggggccagccggccgccccacctggtcttcttgctggcagacgacctaggctggaacgacgtcggcttccacggctcccgcatccgcacgccgcacctggacgcgctggcggccggcggggtgctcctggacaactactacacgcagccgctgtgcacgccgtcgcggagccagctgctcactggccgctaccagatccgtacaggtttacagcaccaaataatctggccctgtcagcccagctgtgttcctctggatgaaaaactcctgccccagctcctaaaagaagcaggttatactacccatatggtcggaaaatggcacctgggaatgtaccggaaagaatgccttccaacccgccgaggatttgatacctactttggatatctcctgggtagtgaagattattattcccatgaacgctgtacattaattgacgctctgaatgtcacacgatgtgctcttgattttcgagatggcgaagaagttgcaacaggatataaaaatatgtattcaacaaacatattcaccaaaagggctatagccctcataactaaccatccaccagagaagcctctgtttctctaccttgctctccagtctgtgcatgagccccttcaggtccctgaggaatacttgaagccatatgactttatccaagacaagaacaggcatcactatgcaggaatggtgtcccttatggatgaagcagtaggaaatgtcactgcagctttaaaaagcagtgggctctggaacaacacggtgttcatcttttctacagataacggagggcagactttggcagggggtaataactggccccttcgaggaagaaaatggagcctgtgggaaggaggcgtccgaggggtgggctttgtggcaagccccttgctgaagcagaagggcgtgaagaaccgggagctcatccacatctctgactggctgccaacactcgtgaagctggccaggggacacaccaatggcacaaagcctctggatggcttcgacgtgtggaaaaccatcagtgaaggaagcccatcccccagaattgagctgctgcataatattgacccgaacttcgtggactcttcaccgtgtcccaggaacagcatggctccagcaaaggatgactcttctcttccagaatattcagcctttaacacatctgtccatgctgcaattagacatggaaattggaaactcctcacgggctacccaggctgtggttactggttccctccaccgtctcaatacaatgtttctgagataccctcatcagacccaccaaccaagaccctctggctctttgatattgatcgggaccctgaagaaagacatgacctgtccagagaatatcctcacatcgtcacaaagctcctgtcccgcctacagttctaccataaacactcagtccccgtgtacttccctgcacaggacccccgctgtgatcccaaggccactggggtgtggggcccttggatgtag

SEQ ID NO: 2 - protein, 533 aa, Homo sapiens
MGPRGAASLPRGPGPRRLLLPVVLPLLLLLLLAPPGSGAGASRPPHLVFLLADDLGWNDVGFHGSRIRTPHLDALAAGGVLLDNYYTQPLCTPSRSQLLTGRYQIRTGLQHQIIWPCQPSCVPLDEKLLPQLLKEAGYTTHMVGKWHLGMYRKECLPTRRGFDTYFGYLLGSEDYYSHERCTLIDALNVTRCALDFRDGEEVATGYKNMYSTNIFTKRAIALITNHPPEKPLFLYLALQSVHEPLQVPEEYLKPYDFIQDKNRHHYAGMVSLMDEAVGNVTAALKSSGLWNNTVFIFSTDNGGQTLAGGNNWPLRGRKWSLWEGGVRGVGFVASPLLKQKGVKNRELIHISDWLPTLVKLARGHTNGTKPLDGFDVWKTISEGSPSPRIELLHNIDPNFVDSSPCPRNSMAPAKDDSSLPEYSAFNTSVHAAIRHGNWKLLTGYPGCGYWFPPPSQYNVSEIPSSDPPTKTLWLFDIDRDPEERHDLSREYPHIVTKLLSRLQFYHKHSVPVYFPAQDPRCDPKATGVWGPWM

SEQ ID NO: 3 - DNA, 3214 bp, Homo sapiens
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcccggagccgccatgggtccgcgcggcgcggcgagcttgccccgaggccccggacctcggcggctgctcctccccgtcgtcctcccgctgctgctgctgctgttgttggcgccgccgggctcgggcgccggggccagccggccgccccacctggtcttcttgctggcagacgacctaggctggaacgacgtcggcttccacggctcccgcatccgcacgccgcacctggacgcgctggcggccggcggggtgctcctggacaactactacacgcagccgctgtgcacgccgtcgcggagccagctgctcactggccgctaccagatccgtacaggtttacagcaccaaataatctggccctgtcagcccagctgtgttcctctggatgaaaaactcctgccccagctcctaaaagaagcaggttatactacccatatggtcggaaaatggcacctgggaatgtaccggaaagaatgccttccaacccgccgaggatttgatacctactttggatatctcctgggtagtgaagattattattcccatgaacgctgtacattaattgacgctctgaatgtcacacgatgtgctcttgattttcgagatggcgaagaagttgcaacaggatataaaaatatgtattcaacaaacatattcaccaaaagggctatagccctcataactaaccatccaccagagaagcctctgtttctctaccttgctctccagtctgtgcatgagccccttcaggtccctgaggaatacttgaagccatatgactttatccaagacaagaacaggcatcactatgcaggaatggtgtcccttatggatgaagcagtaggaaatgtcactgcagctttaaaaagcagtgggctctggaacaacacggtgttcatcttttctacagataacggagggcagactttggcagggggtaataactggccccttcgaggaagaaaatggagcctgtgggaaggaggcgtccgaggggtgggctttgtggcaagccccttgctgaagcagaagggcgtgaagaaccgggagctcatccacatctctgactggctgccaacactcgtgaagctggccaggggacacaccaatggcacaaagcctctggatggcttcgacgtgtggaaaaccatcagtgaaggaagcccatcccccagaattgagctgctgcataatattgacccgaacttcgtggactcttcaccgtgtcccaggaacagcatggctccagcaaaggatgactcttctcttccagaatattcagcctttaacacatctgtccatgctgcaattagacatggaaattggaaactcctcacgggctacccaggctgtggttactggttccctccaccgtctcaatacaatgtttctgagataccctcatcagacccaccaaccaagaccctctggctctttgatattgatcgggaccctgaagaaagacatgacctgtccagagaatatcctcacatcgtcacaaagctcctgtcccgcctacagttctaccataaacactcagtccccgtgtacttccctgcacaggacccccgctgtgatcccaaggccactggggtgtggggcccttggatgtagctcgaatcaagcttatcgattctagtagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 4 - protein, 497 aa, Artificial Sequence
  synthetic
SGAGASRPPHLVFLLADDLGWNDVGFHGSRIRTPHLDALAAGGVLLDNYYTQPLCTPSRSQLLTGRYQIRTGLQHQIIWPCQPSCVPLDEKLLPQLLKEAGYTTHMVGKWHLGMYRKECLPTRRGFDTYFGYLLGSEDYYSHERCTLIDALNVTRCALDFRDGEEVATGYKNMYSTNIFTKRAIALITNHPPEKPLFLYLALQSVHEPLQVPEEYLKPYDFIQDKNRHHYAGMVSLMDEAVGNVTAALKSSGLWNNTVFIFSTDNGGQTLAGGNNWPLRGRKWSLWEGGVRGVGFVASPLLKQKGVKNRELIHISDWLPTLVKLARGHTNGTKPLDGFDVWKTISEGSPSPRIELLHNIDPNFVDSSPCPRNSMAPAKDDSSLPEYSAFNTSVHAAIRHGNWKLLTGYPGCGYWFPPPSQYNVSEIPSSDPPTKTLWLFDIDRDPEERHDLSREYPHIVTKLLSRLQFYHKHSVPVYFPAQDPRCDPKATGVWGPWM

SEQ ID NO: 5 - DNA, 130 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 6 - DNA, 130 bp, Artificial Sequence
  synthetic
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 7 - DNA, 208 bp, Artificial Sequence
  synthetic
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 8 - DNA, 6472 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcccggagccgccatgggtccgcgcggcgcggcgagcttgccccgaggccccggacctcggcggctgctcctccccgtcgtcctcccgctgctgctgctgctgttgttggcgccgccgggctcgggcgccggggccagccggccgccccacctggtcttcttgctggcagacgacctaggctggaacgacgtcggcttccacggctcccgcatccgcacgccgcacctggacgcgctggcggccggcggggtgctcctggacaactactacacgcagccgctgtgcacgccgtcgcggagccagctgctcactggccgctaccagatccgtacaggtttacagcaccaaataatctggccctgtcagcccagctgtgttcctctggatgaaaaactcctgccccagctcctaaaagaagcaggttatactacccatatggtcggaaaatggcacctgggaatgtaccggaaagaatgccttccaacccgccgaggatttgatacctactttggatatctcctgggtagtgaagattattattcccatgaacgctgtacattaattgacgctctgaatgtcacacgatgtgctcttgattttcgagatggcgaagaagttgcaacaggatataaaaatatgtattcaacaaacatattcaccaaaagggctatagccctcataactaaccatccaccagagaagcctctgtttctctaccttgctctccagtctgtgcatgagccccttcaggtccctgaggaatacttgaagccatatgactttatccaagacaagaacaggcatcactatgcaggaatggtgtcccttatggatgaagcagtaggaaatgtcactgcagctttaaaaagcagtgggctctggaacaacacggtgttcatcttttctacagataacggagggcagactttggcagggggtaataactggccccttcgaggaagaaaatggagcctgtgggaaggaggcgtccgaggggtgggctttgtggcaagccccttgctgaagcagaagggcgtgaagaaccgggagctcatccacatctctgactggctgccaacactcgtgaagctggccaggggacacaccaatggcacaaagcctctggatggcttcgacgtgtggaaaaccatcagtgaaggaagcccatcccccagaattgagctgctgcataatattgacccgaacttcgtggactcttcaccgtgtcccaggaacagcatggctccagcaaaggatgactcttctcttccagaatattcagcctttaacacatctgtccatgctgcaattagacatggaaattggaaactcctcacgggctacccaggctgtggttactggttccctccaccgtctcaatacaatgtttctgagataccctcatcagacccaccaaccaagaccctctggctctttgatattgatcgggaccctgaagaaagacatgacctgtccagagaatatcctcacatcgtcacaaagctcctgtcccgcctacagttctaccataaacactcagtccccgtgtacttccctgcacaggacccccgctgtgatcccaaggccactggggtgtggggcccttggatgtagctcgaatcaagcttatcgattctagtagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatcctccggcgttcagcctgtgccacagccgacaggatggtgaccaccatttgccccatatcaccgtcggtactgatcccgtcgtcaataaaccgaaccgctacaccctgagcatcaaactcttttatcagttggatcatgtcggcggtgtcgcggccaagacggtcgagcttcttcaccagaatgacatcaccttcctccaccttcatcctcagcaaatccagcccttcccgatctgttgaactgccggatgccttgtcggtaaagatgcggttagcttttacccctgcatctttgagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacaccatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 9 - DNA, 5612 bp, Artificial Sequence
  synthetic
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 10 - DNA, 477 bp, Artificial Sequence
  synthetic
agggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaga

SEQ ID NO: 11 - DNA, 393 bp, Artificial Sequence
  synthetic
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 12 - DNA, 334 bp, Artificial Sequence
  synthetic
gcatgcttccatgccaaggcccacactgaaatgctcaaatgggagacaaagagattaagctcttatgtaaaatttgctgttttacataactttaatgaatggacaaagtcttgtgcatgggggtgggggtggggttagaggggaacagctccagatggcaaacatacgcaagggatttagtcaaacaactttttggcaaagatggtatgattttgtaatggggtaggaaccaatgaaatgcgaggtaagtatggttaatgatctacagttattggttaaagaagtatattagagcgagtctttctgcacacagatcacctttcctatcaacccc

SEQ ID NO: 13 - DNA, 516 bp, Artificial Sequence
  synthetic
cacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggg

SEQ ID NO: 14 - DNA, 583 bp, Artificial Sequence
  synthetic
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgt

SEQ ID NO: 15 - DNA, 23 bp, Artificial Sequence
  synthetic
tctagttgccagccatctgttgt

SEQ ID NO: 16 - DNA, 18 bp, Artificial Sequence
  synthetic
tgggagtggcaccttcca

SEQ ID NO: 17 - DNA, 21 bp, Artificial Sequence
  synthetic
tcccccgtgccttccttgacc

(19) United States
(12) Patent Application Publication
(10) Pub. No.: US 2019/0343929 A1
(43) Pub. Date: Nov. 14, 2019

(54) COMBINED THERAPY FOR MUCOPOLYSACCHARIDOSIS TYPE VI (MAROTEAUX-LAMY-SYNDROME)

(71) Applicant: FONDAZIONE TELETHON, Rome (RM) (IT)

(72) Inventors: Alberto AURICCHIO, Rome (RM) (IT); Marialuisa ALLIEGRO, Rome (RM) (IT); Rita FERLA, Rome (RM) (IT)

(73) Assignee: FONDAZIONE TELETHON, Rome (RM) (IT)

(21) Appl. No.: 16/331,358

(22) PCT Filed: Sep. 9, 2017
(86) PCT No.: PCT/EP2017/072799

§ 371 Date: Mar. 7, 2019

Related U.S. Application Data
(60) Provisional application No. 62/385,670, filed on Sep. 9, 2016.

(30) Foreign Application Priority Data
Oct. 12, 2016 (DE) 10 2016 219 788.6

(51) Int. Cl. A61K 38/46; A61K 9/00; A61K 48/00

(52) U.S. Cl. CPC A61K 38/465; C12Y 301/06012; A61K 48/0058; A61K 9/0019

(57) ABSTRACT

The present invention relates to a method for the treatment of MPS VI comprising administering an arylsulfatase B by gene therapy to a subject in need thereof, wherein said subject is administered with an arylsulfatase B enzyme replacement therapy (ERT) less frequently than once a week.

### FIELD OF THE INVENTION

The present invention relates to a method for the treatment of MPS VI comprising administering an arylsulfatase B by gene therapy to a subject in need thereof, wherein said subject is also administered with an arylsulfatase B enzyme replacement therapy (ERT) less frequently than once a week.

### BACKGROUND OF THE INVENTION

Lysosomal storage diseases (LSDs) include more than 40 distinct inherited metabolic diseases as autosomal or X-linked recessive. The majority of LSDs are caused by deficient activity of specific lysosomal hydrolases and the progressive accumulation of their substrate(s), which ultimately leads to multisystem cellular and organ dysfunction¹.

In particular, mucolopolysaccharidosis type 6 (MPS VI or Maroteaux-Lamy syndrome; OMIM #253200) is a lysosomal storage disease in which the affected patients lack the enzyme Arylsulfatase B (N-acetylgalactosamine-4-sulfatase, chondroitinsulfatase, chondroitinase, acetylgalactosamine 4-sulfatase, N-acetylgalactosamine 4-sulfate sulfohydrolase, ARSB, ASB), hereinafter ARSB. The enzyme hydrolyses sulfates in the body, by metabolizing the sulfate moiety of glycosaminoglycans (GAGs), which are heterogeneous large sugar molecules in the body⁷⁰. Specifically, ARSB targets two GAGs in particular: dermatan sulfate and chondroitin sulfate.

In the absence of the enzyme, the stepwise degradation of dermatan sulfate is blocked and the substrate accumulates intracellularly in the lysosome in a wide range of tissues.

Lysosomal accumulation of the glycosaminoglycan dermatan sulfate is accompanied by urinary excretion of elevated amounts of the same²⁸. The accumulation of GAGs causes a progressive disorder with multiple organ and tissue involvement in which the infant appears normal at birth, but usually dies before puberty. The diagnosis of MPS VI is usually made at 6-24 months of age when children show progressive deceleration of growth, enlarged liver and spleen, skeletal deformities, coarse facial features, upper airway obstruction, and joint deformities. Progressive clouding of the cornea, communicating hydrocephalus, or heart disease may develop in MPS VI children. Death usually results from respiratory infection or cardiac disease. MPS VI is not typically associated with progressive impairment of mental status, although physical limitations may impact learning and development. Although most MPS VI patients have the severe form of the disease that is usually fatal by the teenage years, affected patients with a less severe form of the disease have been described which may survive for decades.

Lysosomal enzymes are targeted to the lysosomes following binding to the mannose 6-phosphate receptor (Man6PR), but can also be secreted. Extracellular phosphorylated or non-phosphorylated enzyme is taken up by the distal cells via either the Man6PRs or the mannose receptor located on the plasma membrane, and then trafficked to the lysosome². This is the basis for cross-correction of deficient cells through enzyme replacement therapy (ERT), which is currently the standard of care for several LSDs³.

Despite its ability to ameliorate patient outcomes and slow disease progression, the requirement of weekly or bi-weekly (i.e. twice a week) ERT intravenous infusions, which is due to the short plasma half-life of recombinant enzymes^{5, 6}, carries a risk of immune-mediated allergic reactions⁷ and often requires a central venous access, resulting in a low quality of life for the patients. Finally, ERTs are extremely expensive and this represents a barrier for their widespread use in less developed countries^{4,8}. Therefore, there is high need to develop new therapeutic strategies with comparable or better efficacy than ERT, but without the inconvenience of multiple infusions associated to ERT.

Gene therapy is emerging as a successful strategy for the treatment of inherited diseases, including LSDs⁹⁻¹¹. Vectors based on adeno-associated viruses (AAVs) are the most frequently used for in vivo applications of gene therapy, because of their safety profile, wide tropism and ability to provide long-term transgene expression¹².

AAV-mediated gene therapy has been tested successfully in both small and large animal models of LSDs, including Pompe disease, Fabry disease, and mucopolysaccharidoses (MPS)¹³⁻²⁴. In particular, AAV vectors serotype 8 (AAV2/8) are being explored to convert the liver into a factory organ for the systemic release of therapeutic proteins.

A recent clinical trial using intravenous administrations of AAV2/8 in patients with hemophilia B proved the safety and efficacy of AAV2/8 liver gene transfer²⁵, resulting in long-term expression of factor IX (FIX) at therapeutic levels^{26, 27}.

The inventors used a similar approach in animal models of MPS VI and demonstrated that a single systemic administration of AAV2/8 encoding ARSB is able to convert the liver into a source of systemic ARSB.^{13-16, 19}.

However, gene therapy may have some limitations.

In the AAV2/8 clinical trial for hemophilia B, an increase in liver enzymes was observed in subjects receiving a dose of vector of 2×10¹² gc/kg, likely due to cell-mediated immune responses to AAV8, which lead to the elimination of transduced hepatocytes. This increase in liver transaminases was successfully controlled with a short course of glucocorticoids^{26, 27}, however either close monitoring of liver enzymes or prophylactic oral corticosteroids are required to avoid loss of transgene expression.

Additionally, issues have recently arisen concerning the risk of AAV vectors to cause insertional mutagenesis. Integration of vector DNA at highly transcribed loci has been associated with hepatocellular carcinoma (HCC) in mice^{25, 29-33}. More importantly, Chandler and colleagues have demonstrated that HCC incidence is AAV dose-dependent in newborn-injected mice²⁹. Furthermore, a recent study showed insertional mutagenesis due to wild-type AAV2 in human HCC³¹, although a direct role of wild-type AAV2 in human liver tumorigenesis needs to be further investigated. Moreover long term efficacy of gene therapy still needs to be confirmed.

Currently available treatments have shown limited efficacy on some LSDs features, such as those related to bone, brain, cartilage, heart and eye.

Therefore there exists still a need for an improved therapeutically effective management of MPS VI. In particular there is still the need to reduce the safety concerns and costs associated with either gene therapy at high doses (2×10¹² gc/kg) or ERT at frequent regimen (greater than 1 mg/kg weekly injections) and to ensure a long term efficacy of MPS VI treatment.

### SUMMARY OF THE INVENTION

The present invention relates to a combination therapy for MPS VI comprising gene therapy and enzyme replacement therapy (ERT), wherein the ERT is administered less frequently than once a week, less frequently than once every two weeks, less frequently than once every 3 weeks, preferably less frequently than once every 4 weeks, preferably less frequently than once every 8 weeks, preferably less frequently than once every 12 week.

The present invention is based on the surprising finding that a greater reduction of urinary GAGs, considered a sensitive and reliable biomarker of lysosomal storage clearance and therapeutic efficacy, was observed in mice receiving the combined therapy (gene therapy+ERT) when compared to single ERT. Indeed, urinary GAGs were reduced by 59% compared to affected (AF) controls in mice treated with both 2×10¹¹ gc/kg of AAV and ERT than in mice treated with either monthly ERT (82% of AF) or 2×10¹¹ gc/kg of AAV (73% of AF). Likewise, a greater reduction of urinary GAGs was observed in mice receiving both 6×10¹¹ gc/kg of AAV and ERT (41% of AF) compared with mice treated with either ERT (82% of AF) or gene therapy at the same dose (53% of AF)

Similarly, greater reduction of GAG storage in the myocardium and heart valves was observed in mice receiving combined gene and enzyme replacement therapy. The reduction was more consistent in the cohort, which received 6×10¹¹ gc/kg of AAV in combination with ERT, where GAG storage was comparable to NR controls. This is relevant in term of therapeutic efficacy since cardiomyopathy and heart valve involvement are serious complications of MPS VI that often negatively affect its prognosis. Further, published literature indicates cardiac function can be compromised in patients with MPS VI. A leading cause of morbidity and mortality in MPS

VI patients is cardiac disease⁷¹.

Therefore, inventors' data show that gene therapy can be used as a means to rarify ERT administration, reducing both the risks and costs associated with either therapies.

Therefore the present invention provides a combination comprising:
a) a vector comprising a nucleic acid encoding an arylsulfatase B and
b) an arylsulfatase B enzyme replacement therapy (ERT)
   for use in the treatment of MPS VI, wherein the ERT is administered less frequently than once a week.

The present invention also provides a method for the treatment of MPS VI comprising:
a) administering to a subject in need thereof a vector comprising a nucleic acid encoding an arylsulfatase B and
b) administering to said subject an arylsulfatase B enzyme replacement therapy (ERT),
   wherein the ERT is administered less frequently than once a week.

Preferably the nucleic acid encodes a wild-type arylsulfatase B.

Preferably the wild-type arylsulfatase B comprises SEQ ID No. 2 or SEQ ID No. 4.

Preferably the nucleic acid comprises SEQ ID No. 1.

Preferably the nucleic acid is operably linked to a liver-specific promoter.

Preferably the liver-specific promoter is selected from the group consisting of: thyroxine-binding globulin (TBG) promoter, alfa-1-antitripsin promoter, albumin promoter.

Preferably the thyroxine-binding globulin (TBG) promoter comprises SEQ ID No. 11, the alfa-1-antitripsin promoter comprises SEQ ID No. 12 and the albumin promoter comprises SEQ ID No. 13.

Preferably the vector comprises SEQ ID No. 3.

Preferably the vector is selected from the group consisting of: an adenoviral vector, lentiviral vector, retroviral vector, adeno associated vector (AAV) or naked plasmid DNA vector.

Preferably the vector is an adeno-associated viral (AAV) vector.

Preferably the AAV vector is of serotype 8.

Preferably the vector comprises SEQ ID No. 8.

Preferably the dosage of the vector is of from 1×10⁹ to 2×10¹⁶ gc/kg, preferably of from 2×10¹¹ gc/kg to 2×10¹² gc/kg, more preferably is about 6×10¹¹ gc/kg.

Preferably the vector is administered intravenously.

Preferably the arylsulfatase B in the ERT comprises SEQ ID No. 2 or SEQ ID No. 4.

Preferably the arylsulfatase B in the ERT is a recombinant arylsulfatase B.

Preferably the arylsulfatase B in the ERT is administered at a dose range of 0.001 mg/kg to 5 mg/kg, preferably at a dose range of 0.5 mg/kg to 4 mg/kg, more preferably at a dose of 1 mg/kg.

Preferably the arylsulfatase B in the ERT is administered intravenously.

Preferably the arylsulfatase B enzyme replacement therapy is administered less frequently than once every 2 weeks, preferably less frequently than once every 3 weeks, preferably less frequently than once every 4 weeks, preferably less frequently than once every 8 weeks, preferably less frequently than once every 12 weeks.

Preferably the vector is administered at a dose ranging from 2×10¹¹ gc/kg to 2×10¹² gc/kg and the arylsulfatase B enzyme replacement therapy (ERT) is administered at a dose of 1 mg/kg and less frequently than once a week, preferably once a month.

Preferably the vector and the arylsulfatase B enzyme replacement therapy are administered at different times.

Preferably the vector is administered prior to the initiation of the arylsulfatase B enzyme replacement therapy. The vector may be administered few hours (1 to 12 hours) or few days (1 to 5 days) or few months (1 to 6 months) prior to the initiation of the arylsulfatase B enzyme replacement therapy.

Preferably the vector is administered simultaneously with initiation of the arylsulfatase B enzyme replacement therapy.

Preferably the vector is administered only once.

Preferably the vector is administered after the initiation of the arylsulfatase B enzyme replacement therapy. The vector may be administered few hours (1 to 12 hours) or few days (1 to 5 days) or few months (1 to 6 months) after the initiation of the arylsulfatase B enzyme replacement therapy.

### Enzyme Replacement Therapy (ERT)

Enzyme replacement therapy (ERT) involves the systemic administration of natural or recombinantly-derived proteins and/or enzymes to a subject. Approved therapies are typically administered to subjects intravenously and are generally effective in treating the somatic symptoms of the underlying enzyme deficiency. ERT is a treatment replacing an enzyme in cells, e.g. patients cells, in whom that particular enzyme is deficient or absent.

An arylsulfatase B in the precursor form, or a biologically active fragment, variant or analog thereof catalyzes the cleavage of the sulfate ester from terminal N acetylgalactosamine 4-sulfate residues of glycosaminoglycans (GAG), chondroitin 4-sulfate and dermatan sulfate.

“Wild-type” (wt) is a term referring to the natural form, including sequence, of a polynucleotide, polypeptide or protein in a species. A wild-type form is distinguished from a mutant form of a polynucleotide, polypeptide or protein arising from genetic mutation(s).

The term “recombinant” is used herein to refer to recombinant DNA molecules, eg DNA molecules formed by laboratory methods of genetic recombination (such as molecular cloning) to bring together genetic material from multiple sources, creating sequences that would not otherwise be found in the genome. Recombinant is also used to refer to peptides, proteins and entire organisms made using said techniques well known and can be found in the published literature including, for example, in Sambrook et al., Molecular Cloning: A Laboratory Manual (New York: Cold Spring Harbor Laboratory Press, 1989).

The term “variant” means a form having a certain percent sequence identity to the native/wild-type forms.

Variants “functional” or “biologically active”, means that the variant protein is capable of metabolizing glycosaminoglycan dermatan sulfate in vivo.

In one embodiment, a first polypeptide that is an “analog” or “variant” or “derivative” of a second polypeptide is a polypeptide having at least about 50%, 60%, 70%, 80%, 85%, 90%, 95%, 96%, 97%, 98% or 99% sequence homology, but less than 100% sequence homology, with the second polypeptide. Such analogs, variants or derivatives may be comprised of non-naturally occurring amino acid residues, including without limitation, homoarginine, ornithine, penicillamine, and norvaline, as well as naturally occurring amino acid residues.

The terms “identical” and percent “identity”, in the context of two or more polynucleotide or polypeptide sequences, refer to two or more sequences or subsequences that are the same or have a specified percentage of nucleotides or amino acid residues that are the same, when compared and aligned for maximum correspondence, as measured using one of the following sequence comparison algorithms or by visual inspection. In a preferred embodiment, percent identity is determined over the full length of the two nucleic acid or amino acid sequences being compared.

The phrase “substantially homologous” or “substantially identical”, in the context of two nucleic acid or polypeptide sequences, generally refers to two or more sequences or subsequences that have at least 40%, 50%, 60%, 70%, 75%, 80%, 85%, 90%, 95%, or 98% nucleotide or amino acid sequence identity, when compared and aligned for maximum correspondence, as measured using one of the following sequence comparison algorithms or by visual inspection. In certain embodiments, the substantial homology or identity exists over regions of the sequences that are at least about 25, 50, 100 or 150 nucleic acid or amino acid residues in length. In another embodiment, the sequences are substantially homologous or identical over the entire length of either or both comparison sequences.

For sequence comparison, typically one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math., 2: 482 (1981), by the homology alignment algorithm of Needleman & Wunsch, J. Mol. Biol., 48: 443 (1970), by the search for similarity method of Pearson & Lipman, Proc. Natl. Acad. Sci. USA, 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection. One example of a useful algorithm is PILEUP, which uses a simplification of the progressive alignment method of Feng & Doolittle, J. Mol. Evol., 35: 351-360 (1987) and is similar to the method described by Higgins & Sharp, CABIOS, 5: 151-153 (1989). Another algorithm useful for generating multiple alignments of sequences is Clustal W (Thompson et al., Nucleic Acids Research, 22: 4673-4680 (1994)). An example of an algorithm that is suitable for determining percent sequence identity and sequence similarity is the BLAST algorithm (Altschul et al., J. Mol. Biol., 215: 403-410 (1990); Henikoff & Henikoff, Proc. Natl. Acad. Sci. USA, 89: 10915 (1989); Karlin & Altschul, Proc. Natl. Acad. Sci. USA, 90: 5873-5787 (1993)). Software for performing BLAST analyses is publicly available through the National Center for Biotechnology Information.

Preferably, an arylsulfatase B or a precursor form thereof, or a variant, a biological active fragment thereof, or an analog thereof has specific activity in the range of 20-90 units, and more preferably greater than about 50 units per mg protein.

The preferred specific activity of the ARSB according to the present invention is about 20-90 Unit, and more preferably greater than 50 units per milligram protein. Preferably, the enzyme has a deglycosylated weight of about 55 to 56 kDa, most preferably about 55.7 kDa. Preferably, the enzyme has a glycosylated weight of about 63 to 68 kDa, most preferably about 64 to 66 kDa. The present invention also includes biologically active fragments including truncated molecules, analogs and mutants of the naturally-occurring human ARSB.

A variety of parenteral or non-parenteral routes of administration, including oral, transdermal, transmucosal, intrapulmonary (including aerosolized), intramuscular, subcutaneous, or intravenous that deliver equivalent dosages are contemplated. Administration by bolus injection or infusion directly into the joints or CSF is also specifically contemplated, such as intrathecal, intracerebral, intraventricular, via lumbar puncture, or via the cistema magna. A variety of means are known in the art for achieving such intrathecal administration, including pumps, reservoirs, shunts or implants. Preferably the doses are delivered via intravenous infusions lasting 1, 2 or 4 hours, most preferably 4 hours, but may also be delivered by an intravenous bolus.

Preferably, the ERT according to the present invention is administered intravenously over approximately a four-hour period. Also, preferably, it is administered by an intravenous catheter placed in the cephalic or other appropriate vein with an infusion of saline begun at about 30 cc/hr. Further, preferably it is diluted into about 250 cc of normal saline.

The ERT may be administered in a number of ways in addition to the preferred embodiments described above, such as parenteral, topical, intranasal, inhalation or oral administration.

Optionally, the ERT is formulated in a pharmaceutical composition, together with a pharmaceutically-acceptable carrier which may be solid, semi-solid or liquid or an ingestable capsule. Examples of pharmaceutical compositions include tablets, drops such as nasal drops, compositions for topical application such as ointments, jellies, creams and suspensions, aerosols for inhalation, nasal spray, liposomes. Usually the recombinant enzyme comprises between 0.05 and 99% or between 0.5 and 99% by weight of the composition, for example between 0.5 and 20% for compositions intended for injection and between 0.1 and 50% for compositions intended for oral administration.

A particularly preferred method of administering the recombinant enzyme is intravenously. A particularly preferred composition comprises recombinant ARSB, normal saline, phosphate buffer to maintain the pH at about 5-7, and human albumin. The composition may additionally include polyoxyethylenesorbitan, such as polysorbate 20 or 80 (Tween-20 or Tween-80) to improve the stability and prolong shelf life. Alternatively, the composition may include any surfactant or non-ionic detergent known in the art, including but not limited to polyoxyethylene sorbitan 40 or 60; polyoxyethylene fatty acid esters; polyoxyethylene sorbitan monoisostearates; poloxamers, such as poloxamer 188 or poloxamer 407; octoxynol-9 or octoxynol 40.

In a preferred embodiment, the ERT is formulated as 1 mg/mL ARSB in 150 mM NaCl, 10 mM NaPO.sub.4, pH 5.8, 0.005% polysorbate 80.

### Gene Therapy

Gene therapy is the therapeutic delivery of nucleic acid polymers into a patient's cells as a drug to treat disease. Gene therapy comprises administering a vector comprising a nucleic acid encoding an arylsulfatase B.

In particular the nucleic acid comprises SEQ ID No. 1.

Preferably said vector is a vector selected from the group consisting of: adenoviral vectors, lentiviral vectors, retroviral vectors, adeno associated vectors (AAV) or naked plasmid DNA vectors. According to a preferred embodiment, said vector is an adeno-associated virus vector. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). All AAV serotypes apparently exhibit very similar replication properties mediated by homologous rep genes; and all bear three related capsid proteins. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

As used herein, an “AAV vector” refers to nucleic acids, either single-stranded or double-stranded, having an AAV 5′ inverted terminal repeat (ITR) sequence and an AAV 3′ ITR flanking a polynucleotide encoding ARSB operably linked to transcription regulatory elements that are heterologous to the AAV viral genome, i.e., one or more promoters and/or enhancers and, optionally, a polyadenylation sequence and/or one or more introns inserted between exons of the protein-coding sequence.

Such AAV vectors can be replicated and packaged into infectious viral particles when present in a host cell that has been transfected with a vector encoding and expressing rep and cap gene products. A single-stranded AAV vector refers to nucleic acids that are present in the genome of an AAV virus particle, and can be either the sense strand or the anti-sense strand of the nucleic acid sequences disclosed herein. The size of such single-stranded nucleic acids is provided in bases. A double-stranded AAV vector refers to nucleic acids that are present in the DNA of plasmids, e.g., pUC19, or genome of a double-stranded virus, e.g., baculovirus, used to express or transfer the AAV vector nucleic acids. The size of such double-stranded nucleic acids in provided in base pairs (bp). In certain embodiments, AAV vectors of the present invention comprise a nucleic acid sequence encoding a functional ARSB protein.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

AAV “rep” and “cap” genes are genes encoding replication and capsid proteins, respectively. AAV rep and cap genes have been found in all AAV serotypes examined to date, and are described herein and in the references cited. In wild-type AAV, the rep and cap genes are generally found adjacent to each other in the viral genome (i.e., they are “coupled” together as adjoining or overlapping transcriptional units), and they are generally conserved among AAV serotypes. AAV rep and cap genes are also individually and collectively referred to as “AAV packaging genes”. The AAV cap genes in accordance with the present invention encode capsid proteins which are capable of packaging AAV vectors in the presence of rep and adeno helper function and are capable of binding target cellular receptors. In some embodiments, the AAV cap gene encodes a capsid protein having an amino acid sequence derived from a particular AAV serotype, for example the serotypes shown in Table 1.

TABLE 1
AAV serotypes

| AAV Serotype | Genbank Accession No. |
|---|---|
| AAV-1 | NC_002077.1 |
| AAV-2 | NC_001401.2 |
| AAV-3 | NC_001729.1 |
| AAV-3B | AF028705.1 |
| AAV-4 | NC_001829.1 |
| AAV-5 | NC_006152.1 |
| AAV-6 | AF028704.1 |
| AAV-7 | NC_006260.1 |
| AAV-8 | NC_006261.1 |
| AAV-9 | AX753250.1 |
| AAV-10 | AY631965.1 |
| AAV-11 | AY631966.1 |
| AAV-12 | DQ813647.1 |
| AAV-13 | EU285562.1 |

The AAV sequences employed for the production of AAV can be derived from the genome of any AAV serotype. Generally, the AAV serotypes have genomic sequences of significant homology at the amino acid and the nucleic acid levels, provide a similar set of genetic functions, produce virions which are essentially physically and functionally equivalent, and replicate and assemble by practically identical mechanisms. For the genomic sequence of AAV serotypes and a discussion of the genomic similarities see, for example, GenBank Accession number U89790; GenBank Accession number J01901; GenBank Accession number AF043303; GenBank Accession number AF085716; Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000).

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins, Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter. The ITRs employed in the vectors of the present invention may correspond to the same serotype as the associated cap genes, or may differ. In a particularly preferred embodiment, the ITRs employed in the vectors of the present invention correspond to an AAV2 serotype and the cap genes correspond to an AAV8 serotype.

The term “inverted terminal repeat (ITR)” as used herein refers to the art-recognized regions found at the 5′ and 3′ termini of the AAV genome which function in cis as origins of DNA replication and as packaging signals for the viral genome. AAV ITRs, together with the AAV rep coding region, provide for efficient excision and rescue from, and integration of a nucleotide sequence interposed between two flanking ITRs into a host cell genome. Sequences of certain AAV-associated ITRs are disclosed by Yan et al., J. Virol. 79(1):364-379 (2005) which is herein incorporated by reference in its entirety. ITR sequences that find use herein may be full length, wild-type AAV ITRs or fragments thereof that retain functional capability, or may be sequence variants of full-length, wild-type AAV ITRs that are capable of functioning in cis as origins of replication. AAV ITRs useful in the recombinant AAV vectors of the present invention may derive from any known AAV serotype and, in certain preferred embodiments, derive from the AAV2 or AAV8 serotype.

Viral vectors according to the invention may comprise a recombinant nucleic acid operatively linked to transcription regulatory elements.

Viral vectors can be administered directly to patients (in vivo) or they can be used to treat cells in vitro and the modified cells are administered to patients (ex vivo).

A “transcription regulatory element” refers to nucleotide sequences of a gene involved in regulation of genetic transcription including a promoter, plus response elements, activator and enhancer sequences for binding of transcription factors to aid RNA polymerase binding and promote expression, and operator or silencer sequences to which repressor proteins bind to block RNA polymerase attachment and prevent expression. The term “liver specific transcription regulatory element” refers to a regulatory element that modulates gene expression specifically in the liver tissue. Examples of liver specific regulatory elements include, but are not limited to, the mouse thyretin promoter (mTTR), the endogenous human factor VIII promoter (F8), human alpha-1-antitrypsin promoter (hAAT) and active fragments thereof, human albumin minimal promoter, human thyroxine binding globulin (TBG) promoter, and mouse albumin promoter. Enhancers derived from liver specific transcription factor binding sites are also contemplated, such as EBP, DBP, HNF1, HNF3, HNF4, HNF6, with Enhl. Conventional viral and non-viral based delivery methods can be used to introduce nucleic acids polymers into cells (e.g., mammalian cells) and target tissues. Non-viral vector delivery systems include DNA or RNA plasmids, DNA MCs, naked nucleic acid, and nucleic acid complexed with a delivery vehicle such as a liposome or poloxamer. Viral vector delivery systems include DNA and RNA viruses, which have either episomal or integrated genomes after delivery to the cell.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995).

Preferred pharmaceutical carriers depend upon the intended mode of administration of the active agent. Typical modes of administration for protein therapeutics include enteral (e.g., oral) or parenteral (e.g., subcutaneous, intramuscular, intravenous or intraperitoneal injection; or topical, transdermal, or transmucosal administration).

A “pharmaceutically acceptable salt” is a salt that can be formulated into a compound for pharmaceutical use, including but not limited to metal salts (e.g., sodium, potassium, magnesium, calcium, etc.) and salts of ammonia or organic amines.

By “pharmaceutically acceptable” or “pharmacologically acceptable” is meant a material that is not biologically or otherwise undesirable, i.e., the material may be administered to an individual without causing any undesirable biological effects or without interacting in a deleterious manner with any of the components of the composition in which it is contained or with any components present on or in the body of the individual.

As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. The term does not denote a particular age or gender.

The term “effective amount” means a dosage sufficient to produce a desired result on a health condition, pathology, or disease of a subject or for a diagnostic purpose. The desired result may comprise a subjective or objective improvement in the recipient of the dosage. “Therapeutically effective amount” refers to that amount of an agent effective to produce the intended beneficial effect on health. An appropriate “effective” amount in any individual case may be determined by one of ordinary skill in the art using routine experimentation. It will be understood that the specific dose level and frequency of dosage for any particular patient may be varied and will depend upon a variety of factors, including the activity of the specific compound employed; the bioavailability, metabolic stability, rate of excretion and length of action of that compound; the mode and time of administration of the compound; the age, body weight, general health, sex, and diet of the patient; and the severity of the particular condition. “Treatment” refers to prophylactic treatment or therapeutic treatment. In certain embodiments, “treatment” refers to administration of a compound or composition to a subject for therapeutic or prophylactic purposes.

A “prophylactic” treatment is a treatment administered to a subject who does not exhibit signs of a disease or exhibits only early signs of the disease, for the purpose of decreasing the risk of developing pathology. The compounds or compositions of the disclosure may be given as a prophylactic treatment to reduce the likelihood of developing a pathology or to minimize the severity of the pathology, if developed.

A “therapeutic” treatment is a treatment administered to a subject who exhibits signs or symptoms of pathology for the purpose of diminishing or eliminating those signs or symptoms. The signs or symptoms may be biochemical, cellular, histological, functional or physical, subjective or objective. The compounds of the disclosure may also be given as a therapeutic treatment.

Deficiency in ARSB activity can be observed, e.g., as activity levels of 50% or less, 25% or less, or 10% or less compared, to normal levels of ARSB activity and can manifest as a mucopolysaccharidosis, for example mucopolysaccharidosis VI (MPS VI) or Maroteaux-Lamy syndrome.

A therapy according to the invention displays therapeutically efficacy when it provides a beneficial effect in the human patient and preferably provides improvements in any one of the following: joint mobility, pain, or stiffness, either subjectively or objectively; exercise tolerance or endurance, for example, as measured by walking or climbing ability; pulmonary function, for example, as measured by FVC, FEV.sub.1 or FET; cardiac function, for example, as measured by ventricular hypertrophy, valve obstruction, or valve regurgitation; visual acuity; or activities of daily living, for example, as measured by ability to stand up from sitting, pull clothes on or off, or pick up small objects.

Preferably, therapeutic efficacy of treatment is displayed by reduction in urinary GAG excretions of at least 20%, at least 30%, at least 40%, at least 50%, at least 80%, at least 90% compared to urinary GAG excretion prior to treatment.

A combination therapy is a therapeutic intervention in which more than one therapy is administered to the subject.

The precise dose and schedule of administration will depend on the stage and severity of the condition, and the individual characteristics of the patient being treated, as well as the most effective biological activity of treatment as will be appreciated by one of ordinary skill in the art. It is also contemplated that the treatment continues until satisfactory results are observed, which can be as soon as after 1 cycle although from about 3 to about 6 cycles or more cycles may be required such as in a maintenance schedule of administration.

The exact amount, frequency and period of administration of the ERT or gene therapy of the present invention will vary, of course, depending upon the sex, age and medical condition of the patient as well as the severity and type of the disease as determined by the attending clinician.

The schedule of treatment with the combination can foresee that the ERT is administered concomitantly, before and/or after the gene therapy identified above. Interval between ERT and gene therapy may vary from days to weeks.

Still further aspects include combining the therapy described herein with other therapies for synergistic or additive benefit.

According to preferred embodiments, ERT is administered at least once.

In a preferred embodiment the combination produces a diminution in glycosaminoglycans (GAG) levels.

Preferably, the dosage of the vector for gene therapy is of from 1×10⁹ to 2×10¹⁶ gc/kg, preferably of from 2×10¹⁰ gc/kg to 2×10¹⁴ gc/kg, preferably of from 2×10¹¹ gc/kg to 2×10¹² gc/kg, more preferably is about 6×10¹¹ gc/kg. In the present invention a preferred dose of enzyme is 1 mg/kg. In the present invention a preferred dose of vector is less than 2×10¹² gc/kg, preferably about 6×10¹¹ gc/kg.

In the present invention glycosaminoglycans levels in urine and tissues may be measured by any known method in the art for instance as described in^{72, 73} and in the material and method section below.

### DETAILED DESCRIPTION OF THE INVENTION

### Figures

FIG. 1. Serum ARSB levels in mice receiving gene therapy and/or monthly ERT. Serum ARSB (pg/ml) was monitored up to 210 days of age. Serum samples were collected monthly and before ERT administration in mice receiving ERT with or without gene therapy. Each bar represents the mean±SE of serum ARSB levels and the corresponding value is indicated above each bar. Serum ARSB levels were undetectable in affected controls (data not shown). Values of serum ARSB levels (mean±SE) in normal mice (NR) are shown in the figure. Number (n) of animals is: NR, n=23 at post-natal day 30 and 150, n=24 at post-natal day 60, 90 and 120, n=22 at post-natal day 180 and n=16 at post-natal day 210; ERT, n=4; AAV 2×10¹¹ gc/kg, n=5; AAV 2×10¹¹+ERT, n=7 except for post-natal day 210 (n=4); AAV 6×10¹¹ gc/kg, n=4; AAV 6×10¹¹ gc/kg+ERT, n=5 except for post-natal day 210 (n=3). The lower number of values in the later than earlier time points is due to animal sacrifice, which varied between days 180 and 210 of age. Statistical comparisons were made using the one-way ANOVA and the Tukey post hoc test. The exact p values obtained are indicated in the Material and Methods section. Abbreviations: AAV, adeno-associated viral vector AAV2/8.TBG.hARSB; ERT, enzyme replacement therapy.

FIG. 2. Reduction of urinary GAGs in mice receiving gene therapy and/or monthly ERT. Urinary GAGs were measured monthly in treated MPS VI mice (gray bars), in normal (NR, white bars) and affected (AF, black bars) controls. Urinary GAG levels measured were averaged for all animals within the same group of treatment and for all time points and the resulting value is reported as a percentage (%) of age-matched AF controls, as indicated inside each bar. Results are represented as mean±SE. Number (n) of animals is: NR, n=39 at post-natal day 60 and 90, n=34 at post-natal day 120, n=31 at post-natal day 150, n=26 at post-natal day 180 and n=21 at post-natal day 210; AF, n=9; ERT, n=5 except for post-natal day 90, 150 and 180 (n=4); AAV 2×10¹¹, n=6; AAV 2×10¹¹+ERT, n=8 except for post-natal day 210 (n=5); AAV 6×10¹¹, n=5 except for post-natal day 210 (n=4); AAV 6×10¹¹+ERT, n=5 except for post-natal day 210 (n=3). The lower number of values in the later than earlier time points is due to either technical challenges in the collection of samples when too numerous or to animal sacrifice, which varied between days 180 and 210 of age. Statistical comparisons were made using the one-way ANOVA and the Tukey post hoc test. The p value is: *<0.05 and **<<0.01; the p-value of AF vs. all groups is: ^{°°}<<0.01. The exact p-values obtained are indicated in the Material and Methods section. Abbreviations: AAV, AAV2/8.TBG.hARSB; ERT, monthly ERT.

FIG. 3. Reduction of urinary GAGs in mice receiving gene therapy and/or monthly ERT. Urinary GAGs were measured in treated MPS VI mice (gray bars), in normal (NR, white bars) and in affected (AF, black bars) controls. Urinary GAG levels measured at each time point were averaged for all animals within the same group of treatment and the resulting value is reported as a percentage (%) of age-matched AF controls, as indicated above each bar. Results are represented as mean±SE. Number (n) of animals is: NR, n=39 at post-natal day 60 and 90, n=34 at post-natal day 120, n=31 at post-natal day 150, n=27 at post-natal day 180 and n=21 at post-natal day 210; AF, n=9; ERT, n=5 except for post-natal day 90, 150 and 180 (n=4); AAV 2×10¹¹, n=6; AAV 2×10¹¹+ERT, n=8 except for post-natal day 210 (n=5); AAV 6×10¹¹, n=5 except for post-natal day 210 (n=4); AAV 6×10¹¹+ERT, n=5 except for post-natal day 210 (n=3). The lower number of values in the later than earlier time points is due to either technical challenges in the collection of samples when too numerous or to animal sacrifice, which varied between days 180 and 210 of age. Statistical comparisons were made using the one-way ANOVA and the Tukey post hoc test. The p value vs. AF is: * <0.05 and ** <0.01. The exact p values obtained are indicated in the Material and Methods section. Abbreviations: AAV, AAV2/8.TBG.hARSB; ERT: monthly ERT.

FIG. 4. Alcian blue staining in the liver, kidney and spleen of mice receiving gene therapy and/or monthly ERT. Reduction of GAGs storage in the liver, kidney and spleen was also evaluated by Alcian blue staining of histological sections obtained from MPS VI mice receiving AAV and/or monthly ERT and from normal (NR) and affected (AF) mice. All MPS VI treated mice that were sacrificed between days 180 and 210 of age were included in the histological analysis. Representative images are shown. Scale bar is 40 μm (magnification is 20×).

FIG. 5. Reduction of GAG storage in the heart valves and myocardium of mice receiving gene therapy and/or monthly ERT. Reduction of GAGs storage in the heart valves and myocardium was evaluated by Alcian blue staining of histological sections obtained from MPS VI mice receiving AAV.TBG.hARSB (AAV) and/or monthly ERT (ERT) and from normal (NR) and affected (AF) controls. All MPS VI treated mice that were sacrificed between days 180 and 210 of age were included in the histological analysis. Representative images are shown. A scale bar is indicated inside the figure (magnification is 40×). Alcian blue staining was quantified as a measure of GAGs storage in heart valves and myocardium. Specifically, Alcian Blue was quantified using the Image J software by measuring RGB intensity on images of histological sections. Results are reported inside each representative image and in the relative histogram OF FIGS. 8 and 9 as mean±SE. Number (n) of animals is: NR, n=3, AF, N=3, ERT, N=3, AAV 2×10¹¹, n=4; AAV 2×10¹¹+ERT, n=5, AAV 6×10¹¹, n=3; AAV 6×10¹¹+ERT, n=3. Statistical comparisons were made using the one-way ANOVA and the Tukey post hoc test. The p value vs. AF is: ** <<0.01. The exact p values obtained are indicated in the Material and Methods section.

FIG. 6. Reduction of liver and kidney GUSB activity in mice receiving gene therapy and/or monthly ERT.

Beta-glucuronidase (GUSB) activity was measured in liver (a) and kidney (b) of treated MPS VI mice (gray bars), and of normal (NR, white bars) and affected (AF, black bars) controls. GUSB activity was averaged for all animals within the same group of treatment and the resulting value is reported as mean±SE. The number of animals is 5 per each group. Statistical comparisons were made using the one-way ANOVA and the Tukey post hoc test. The p value vs AF is: *<0.05 and **<<0.01. The exact p-values obtained are indicated in the Material and Methods section. Abbreviations: AAV, AAV2/8.TBG.hARSB; ERT, monthly ERT.

FIG. 7. Map of vector used for gene therapy in the examples, according to a preferred embodiment of the invention.

FIG. 8. Histogram representing results of Alcian blu quantification of FIG. 5 in heart valves.

FIG. 9. Histogram representing results of Alcian blu quantification of FIG. 5 in myocardium

SEQUENCES

SEQ ID No. 1 hARSB polynucleotide coding sequence
ATGGGTCCGCGCGGCGCGGCGAGCTTGCCCCGAGGCCCCGGACCTCGGCGGCTGCTCCTCCCCGTCGTCCTCCCGC
TGCTGCTGCTGCTGTTGTTGGCGCCGCCGGGCTCGGGCGCCGGGGCCAGCCGGCCGCCCCACCTGGTCTTCTTGCT
GGCAGACGACCTAGGCTGGAACGACGTCGGCTTCCACGGCTCCCGCATCCGCACGCCGCACCTGGACGCGCTGGCG
GCCGGCGGGGTGCTCCTGGACAACTACTACACGCAGCCGCTGTGCACGCCGTCGCGGAGCCAGCTGCTCACTGGCC
GCTACCAGATCCGTACAGGTTTACAGCACCAAATAATCTGGCCCTGTCAGCCCAGCTGTGTTCCTCTGGATGAAAA
ACTCCTGCCCCAGCTCCTAAAAGAAGCAGGTTATACTACCCATATGGTCGGAAAATGGCACCTGGGAATGTACCGG
AAAGAATGCCTTCCAACCCGCCGAGGATTTGATACCTACTTTGGATATCTCCTGGGTAGTGAAGATTATTATTCCC
ATGAACGCTGTACATTAATTGACGCTCTGAATGTCACACGATGTGCTCTTGATTTTCGAGATGGCGAAGAAGTTGC
AACAGGATATAAAAATATGTATTCAACAAACATATTCACCAAAAGGGCTATAGCCCTCATAACTAACCATCCACCA
GAGAAGCCTCTGTTTCTCTACCTTGCTCTCCAGTCTGTGCATGAGCCCCTTCAGGTCCCTGAGGAATACTTGAAGC
CATATGACTTTATCCAAGACAAGAACAGGCATCACTATGCAGGAATGGTGTCCCTTATGGATGAAGCAGTAGGAAA
TGTCACTGCAGCTTTAAAAAGCAGTGGGCTCTGGAACAACACGGTGTTCATCTTTTCTACAGATAACGGAGGGCAG
ACTTTGGCAGGGGGTAATAACTGGCCCCTTCGAGGAAGAAAATGGAGCCTGTGGGAAGGAGGCGTCCGAGGGGTGG
GCTTTGTGGCAAGCCCCTTGCTGAAGCAGAAGGGCGTGAAGAACCGGGAGCTCATCCACATCTCTGACTGGCTGCC
AACACTCGTGAAGCTGGCCAGGGGACACACCAATGGCACAAAGCCTCTGGATGGCTTCGACGTGTGGAAAACCATC
AGTGAAGGAAGCCCATCCCCCAGAATTGAGCTGCTGCATAATATTGACCCGAACTTCGTGGACTCTTCACCGTGTC
CCAGGAACAGCATGGCTCCAGCAAAGGATGACTCTTCTCTTCCAGAATATTCAGCCTTTAACACATCTGTCCATGC
TGCAATTAGACATGGAAATTGGAAACTCCTCACGGGCTACCCAGGCTGTGGTTACTGGTTCCCTCCACCGTCTCAA
TACAATGTTTCTGAGATACCCTCATCAGACCCACCAACCAAGACCCTCTGGCTCTTTGATATTGATCGGGACCCTG
AAGAAAGACATGACCTGTCCAGAGAATATCCTCACATCGTCACAAAGCTCCTGTCCCGCCTACAGTTCTACCATAA
ACACTCAGTCCCCGTGTACTTCCCTGCACAGGACCCCCGCTGTGATCCCAAGGCCACTGGGGTGTGGGGCCCTTGG
ATGTAG
SEQ ID No. 2 human ARSB polypeptide sequence
MGPRGAASLPRGPGPRRLLLPVVLPLLLLLLLAPPGSGAGASRPPHLVFLLADDLGWNDVGFHGSRIRTPHLDALA
AGGVLLDNYYTQPLCTPSRSQLLTGRYQIRTGLQHQIIWPCQPSCVPLDEKLLPQLLKEAGYTTHMVGKWHLGMYR
KECLPTRRGFDTYFGYLLGSEDYYSHERCTLIDALNVTRCALDFRDGEEVATGYKNMYSTNIFTKRAIALITNHPP
EKPLFLYLALQSVHEPLQVPEEYLKPYDFIQDKNRHHYAGMVSLMDEAVGNVTAALKSSGLWNNTVFIFSTDNGGQ
TLAGGNNWPLRGRKWSLWEGGVRGVGFVASPLLKQKGVKNRELIHISDWLPTLVKLARGHTNGTKPLDGFDVWKTI
SEGSPSPRIELLHNIDPNFVDSSPCPRNSMAPAKDDSSLPEYSAFNTSVHAAIRHGNWKLLTGYPGCGYWFPPPSQ
YNVSEIPSSDPPTKTLWLFDIDRDPEERHDLSREYPHIVTKLLSRLQFYHKHSVPVYFPAQDPRCDPKATGVWGPW
M
SEQ ID No. 3 hARSB expression cassette
CTGCGCGCTCGCTCGCTCACTGAGGCCGCCCGGGCAAAGCCCGGGCGTCGGGCGACCTTTGGTCGCCCGGCCTCAG
TGAGCGAGCGAGCGCGCAGAGAGGGAGTGGCCAACTCCATCACTAGGGGTTCCTTGTAGTTAATGATTAACCCGCC
ATGCTACTTATCTACCAGGGTAATGGGGATCCTCTAGAACTATAGCTAGAATTCGCCCTTAAGCTAGCAGGTTAAT
TTTTAAAAAGCAGTCAAAAGTCCAAGTGGCCCTTGGCAGCATTTACTCTCTCTGTTTGCTCTGGTTAATAATCTCA
GGAGCACAAACATTCCAGATCCAGGTTAATTTTTAAAAAGCAGTCAAAAGTCCAAGTGGCCCTTGGCAGCATTTAC
TCTCTCTGTTTGCTCTGGTTAATAATCTCAGGAGCACAAACATTCCAGATCCGGCGCGCCAGGGCTGGAAGCTACC
TTTGACATCATTTCCTCTGCGAATGCATGTATAATTTCTACAGAACCTATTAGAAAGGATCACCCAGCCTCTGCTT
TTGTACAACTTTCCCTTAAAAAACTGCCAATTCCACTGCTGTTTGGCCCAATAGTGAGAACTTTTTCCTGCTGCCT
CTTGGTGCTTTTGCCTATGGCCCCTATTCTGCCTGCTGAAGACACTCTTGCCAGCATGGACTTAAACCCCTCCAGC
TCTGACAATCCTCTTTCTCTTTTGTTTTACATGAAGGGTCTGGCAGCCAAAGCAATCACTCAAAGTTCAAACCTTA
TCATTTTTTGCTTTGTTCCTCTTGGCCTTGGTTTTGTACATCAGCTTTGAAAATACCATCCCAGGGTTAATGCTGG
GGTTAATTTATAACTAAGAGTGCTCTAGTTTTGCAATACAGGACATGCTATAAAAATGGAAAGATGTTGCTTTCTG
AGAGACTGCAGAAGTTGGTCGTGAGGCACTGGGCAGGTAAGTATCAAGGTTACAAGACAGGTTTAAGGAGACCAAT
AGAAACTGGGCTTGTCGAGACAGAGAAGACTCTTGCGTTTCTGATAGGCACCTATTGGTCTTACTGACATCCACTT
TGCCTTTCTCTCCACAGGTGTCCAGGCCCGGAGCCGCCATGGGTCCGCGCGGCGCGGCGAGCTTGCCCCGAGGCCC
CGGACCTCGGCGGCTGCTCCTCCCCGTCGTCCTCCCGCTGCTGCTGCTGCTGTTGTTGGCGCCGCCGGGCTCGGGC
GCCGGGGCCAGCCGGCCGCCCCACCTGGTCTTCTTGCTGGCAGACGACCTAGGCTGGAACGACGTCGGCTTCCACG
GCTCCCGCATCCGCACGCCGCACCTGGACGCGCTGGCGGCCGGCGGGGTGCTCCTGGACAACTACTACACGCAGCC
GCTGTGCACGCCGTCGCGGAGCCAGCTGCTCACTGGCCGCTACCAGATCCGTACAGGTTTACAGCACCAAATAATC
TGGCCCTGTCAGCCCAGCTGTGTTCCTCTGGATGAAAAACTCCTGCCCCAGCTCCTAAAAGAAGCAGGTTATACTA
CCCATATGGTCGGAAAATGGCACCTGGGAATGTACCGGAAAGAATGCCTTCCAACCCGCCGAGGATTTGATACCTA
CTTTGGATATCTCCTGGGTAGTGAAGATTATTATTCCCATGAACGCTGTACATTAATTGACGCTCTGAATGTCACA
CGATGTGCTCTTGATTTTCGAGATGGCGAAGAAGTTGCAACAGGATATAAAAATATGTATTCAACAAACATATTCA
CCAAAAGGGCTATAGCCCTCATAACTAACCATCCACCAGAGAAGCCTCTGTTTCTCTACCTTGCTCTCCAGTCTGT
GCATGAGCCCCTTCAGGTCCCTGAGGAATACTTGAAGCCATATGACTTTATCCAAGACAAGAACAGGCATCACTAT
GCAGGAATGGTGTCCCTTATGGATGAAGCAGTAGGAAATGTCACTGCAGCTTTAAAAAGCAGTGGGCTCTGGAACA
ACACGGTGTTCATCTTTTCTACAGATAACGGAGGGCAGACTTTGGCAGGGGGTAATAACTGGCCCCTTCGAGGAAG
AAAATGGAGCCTGTGGGAAGGAGGCGTCCGAGGGGTGGGCTTTGTGGCAAGCCCCTTGCTGAAGCAGAAGGGCGTG
AAGAACCGGGAGCTCATCCACATCTCTGACTGGCTGCCAACACTCGTGAAGCTGGCCAGGGGACACACCAATGGCA
CAAAGCCTCTGGATGGCTTCGACGTGTGGAAAACCATCAGTGAAGGAAGCCCATCCCCCAGAATTGAGCTGCTGCA
TAATATTGACCCGAACTTCGTGGACTCTTCACCGTGTCCCAGGAACAGCATGGCTCCAGCAAAGGATGACTCTTCT
CTTCCAGAATATTCAGCCTTTAACACATCTGTCCATGCTGCAATTAGACATGGAAATTGGAAACTCCTCACGGGCT
ACCCAGGCTGTGGTTACTGGTTCCCTCCACCGTCTCAATACAATGTTTCTGAGATACCCTCATCAGACCCACCAAC
CAAGACCCTCTGGCTCTTTGATATTGATCGGGACCCTGAAGAAAGACATGACCTGTCCAGAGAATATCCTCACATC
GTCACAAAGCTCCTGTCCCGCCTACAGTTCTACCATAAACACTCAGTCCCCGTGTACTTCCCTGCACAGGACCCCC
GCTGTGATCCCAAGGCCACTGGGGTGTGGGGCCCTTGGATGTAGCTCGAATCAAGCTTATCGATTCTAGTAGATCT
GCCTCGACTGTGCCTTCTAGTTGCCAGCCATCTGTTGTTTGCCCCTCCCCCGTGCCTTCCTTGACCCTGGAAGGTG
CCACTCCCACTGTCCTTTCCTAATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGGCATGCTGGGGACTCGAGTTAAGGG
CGAATTCCCGATAAGGATCTTCCTAGAGCATGGCTACAATTCGTTGATCTGAATTTCGACCACCCATAATACCCAT
TACCCTGGTAGATAAGTAGCATGGCGGGTTAATCATTAACTACAAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTGCCCGGGCGGCCT
CAGTGAGCGAGCGAGCGCGCAG
Legend
5′ ITR: nt 1-130
3′ ITR: nt 3085-3214
Human thyroxine-binding globulin (TBG) promoter: nt 220-917 (of
which AMBP enhancer (alpha-1-microglobulin/bikunin precursor)
gene transcription regulatory region,: nt 220-426)
Intron: nt 950-1081
Kozak sequence: nt 1089-1102
hARSB Coding sequence: nt 1103-2704
Bovine Growth Hormone (BGH) polyA: nt 2744-2951
SEQ ID No. 4 galsulfase
SGAGASRPPHLVFLLADDLGWNDVGFHGSRIRTPHLDALAAGGVLLDNYYTQPLCTPSRS
QLLTGRYQIRTGLQHQIIWPCQPSCVPLDEKLLPQLLKEAGYTTHMVGKWHLGMYRKECL
PTRRGFDTYFGYLLGSEDYYSHERCTLIDALNVTRCALDFRDGEEVATGYKNMYSTNIFT
KRAIALITNHPPEKPLFLYLALQSVHEPLQVPEEYLKPYDFIQDKNRHHYAGMVSLMDEA
VGNVTAALKSSGLWNNTVFIFSTDNGGQTLAGGNNWPLRGRKWSLWEGGVRGVGFVASPL
LKQKGVKNRELIHISDWLPTLVKLARGHTNGTKPLDGFDVWKTISEGSPSPRIELLHNID
PNFVDSSPCPRNSMAPAKDDSSLPEYSAFNTSVHAAIRHGNWKLLTGYPGCGYWFPPPSQ
YNVSEIPSSDPPTKTLWLFDIDRDPEERHDLSREYPHIVTKLLSRLQFYHKHSVPVYFPA
QDPRCDPKATGVWGPWM
SEQ ID No. 5 5′ ITR
CTGCGCGCTCGCTCGCTCACTGAGGCCGCCCGGGCAAAGCCCGGGCGTCGGGCGACCTTTGGTCGCCCGGCCTCAG
TGAGCGAGCGAGCGCGCAGAGAGGGAGTGGCCAACTCCATCACTAGGGGTTCCT
SEQ ID No. 6 3′ ITR
AGGAACCCCTAGTGATGGAGTTGGCCACTCCCTCTCTGCGCGCTCGCTCGCTCACTGAGGCCGGGCGACCAAAGGT
CGCCCGACGCCCGGGCTTTGCCCGGGCGGCCTCAGTGAGCGAGCGAGCGCGCAG
SEQ ID No. 7 BGH polyA sequence
CTGTGCCTTCTAGTTGCCAGCCATCTGTTGTTTGCCCCTCCCCCGTGCCTTCCTTGACCCTGGAAGGTGCCACTCC
CACTGTCCTTTCCTAATAAAATGAGGAAATTGCATCGCATTGTCTGAGTAGGTGTCATTCTATTCTGGGGGGTGGG
GTGGGGCAGGACAGCAAGGGGGAGGATTGGGAAGACAATAGCAGGCATGCTGGGGA
SEQ ID No. 8 pAAV2.1.TBG-hARSB
CTGCGCGCTCGCTCGCTCACTGAGGCCGCCCGGGCAAAGCCCGGGCGTCGGGCGACCTTTGGTCGCCCGGCCTCAG
TGAGCGAGCGAGCGCGCAGAGAGGGAGTGGCCAACTCCATCACTAGGGGTTCCTTGTAGTTAATGATTAACCCGCC
ATGCTACTTATCTACCAGGGTAATGGGGATCCTCTAGAACTATAGCTAGAATTCGCCCTTAAGCTAGCAGGTTAAT
TTTTAAAAAGCAGTCAAAAGTCCAAGTGGCCCTTGGCAGCATTTACTCTCTCTGTTTGCTCTGGTTAATAATCTCA
GGAGCACAAACATTCCAGATCCAGGTTAATTTTTAAAAAGCAGTCAAAAGTCCAAGTGGCCCTTGGCAGCATTTAC
TCTCTCTGTTTGCTCTGGTTAATAATCTCAGGAGCACAAACATTCCAGATCCGGCGCGCCAGGGCTGGAAGCTACC
TTTGACATCATTTCCTCTGCGAATGCATGTATAATTTCTACAGAACCTATTAGAAAGGATCACCCAGCCTCTGCTT
TTGTACAACTTTCCCTTAAAAAACTGCCAATTCCACTGCTGTTTGGCCCAATAGTGAGAACTTTTTCCTGCTGCCT
CTTGGTGCTTTTGCCTATGGCCCCTATTCTGCCTGCTGAAGACACTCTTGCCAGCATGGACTTAAACCCCTCCAGC
TCTGACAATCCTCTTTCTCTTTTGTTTTACATGAAGGGTCTGGCAGCCAAAGCAATCACTCAAAGTTCAAACCTTA
TCATTTTTTGCTTTGTTCCTCTTGGCCTTGGTTTTGTACATCAGCTTTGAAAATACCATCCCAGGGTTAATGCTGG
GGTTAATTTATAACTAAGAGTGCTCTAGTTTTGCAATACAGGACATGCTATAAAAATGGAAAGATGTTGCTTTCTG
AGAGACTGCAGAAGTTGGTCGTGAGGCACTGGGCAGGTAAGTATCAAGGTTACAAGACAGGTTTAAGGAGACCAAT
AGAAACTGGGCTTGTCGAGACAGAGAAGACTCTTGCGTTTCTGATAGGCACCTATTGGTCTTACTGACATCCACTT
TGCCTTTCTCTCCACAGGTGTCCAGGCCCGGAGCCGCCATGGGTCCGCGCGGCGCGGCGAGCTTGCCCCGAGGCCC
CGGACCTCGGCGGCTGCTCCTCCCCGTCGTCCTCCCGCTGCTGCTGCTGCTGTTGTTGGCGCCGCCGGGCTCGGGC
GCCGGGGCCAGCCGGCCGCCCCACCTGGTCTTCTTGCTGGCAGACGACCTAGGCTGGAACGACGTCGGCTTCCACG
GCTCCCGCATCCGCACGCCGCACCTGGACGCGCTGGCGGCCGGCGGGGTGCTCCTGGACAACTACTACACGCAGCC
GCTGTGCACGCCGTCGCGGAGCCAGCTGCTCACTGGCCGCTACCAGATCCGTACAGGTTTACAGCACCAAATAATC
TGGCCCTGTCAGCCCAGCTGTGTTCCTCTGGATGAAAAACTCCTGCCCCAGCTCCTAAAAGAAGCAGGTTATACTA
CCCATATGGTCGGAAAATGGCACCTGGGAATGTACCGGAAAGAATGCCTTCCAACCCGCCGAGGATTTGATACCTA
CTTTGGATATCTCCTGGGTAGTGAAGATTATTATTCCCATGAACGCTGTACATTAATTGACGCTCTGAATGTCACA
CGATGTGCTCTTGATTTTCGAGATGGCGAAGAAGTTGCAACAGGATATAAAAATATGTATTCAACAAACATATTCA
CCAAAAGGGCTATAGCCCTCATAACTAACCATCCACCAGAGAAGCCTCTGTTTCTCTACCTTGCTCTCCAGTCTGT
GCATGAGCCCCTTCAGGTCCCTGAGGAATACTTGAAGCCATATGACTTTATCCAAGACAAGAACAGGCATCACTAT
GCAGGAATGGTGTCCCTTATGGATGAAGCAGTAGGAAATGTCACTGCAGCTTTAAAAAGCAGTGGGCTCTGGAACA
ACACGGTGTTCATCTTTTCTACAGATAACGGAGGGCAGACTTTGGCAGGGGGTAATAACTGGCCCCTTCGAGGAAG
AAAATGGAGCCTGTGGGAAGGAGGCGTCCGAGGGGTGGGCTTTGTGGCAAGCCCCTTGCTGAAGCAGAAGGGCGTG
AAGAACCGGGAGCTCATCCACATCTCTGACTGGCTGCCAACACTCGTGAAGCTGGCCAGGGGACACACCAATGGCA
CAAAGCCTCTGGATGGCTTCGACGTGTGGAAAACCATCAGTGAAGGAAGCCCATCCCCCAGAATTGAGCTGCTGCA
TAATATTGACCCGAACTTCGTGGACTCTTCACCGTGTCCCAGGAACAGCATGGCTCCAGCAAAGGATGACTCTTCT
CTTCCAGAATATTCAGCCTTTAACACATCTGTCCATGCTGCAATTAGACATGGAAATTGGAAACTCCTCACGGGCT
ACCCAGGCTGTGGTTACTGGTTCCCTCCACCGTCTCAATACAATGTTTCTGAGATACCCTCATCAGACCCACCAAC
CAAGACCCTCTGGCTCTTTGATATTGATCGGGACCCTGAAGAAAGACATGACCTGTCCAGAGAATATCCTCACATC
GTCACAAAGCTCCTGTCCCGCCTACAGTTCTACCATAAACACTCAGTCCCCGTGTACTTCCCTGCACAGGACCCCC
GCTGTGATCCCAAGGCCACTGGGGTGTGGGGCCCTTGGATGTAGCTCGAATCAAGCTTATCGATTCTAGTAGATCT
GCCTCGACTGTGCCTTCTAGTTGCCAGCCATCTGTTGTTTGCCCCTCCCCCGTGCCTTCCTTGACCCTGGAAGGTG
CCACTCCCACTGTCCTTTCCTAATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGGCATGCTGGGGACTCGAGTTAAGGG
CGAATTCCCGATAAGGATCTTCCTAGAGCATGGCTACAATTCGTTGATCTGAATTTCGACCACCCATAATACCCAT
TACCCTGGTAGATAAGTAGCATGGCGGGTTAATCATTAACTACAAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTGCCCGGGCGGCCT
CAGTGAGCGAGCGAGCGCGCAGCCTTAATTAACCTAATTCACTGGCCGTCGTTTTACAACGTCGTGACTGGGAAAA
CCCTGGCGTTACCCAACTTAATCGCCTTGCAGCACATCCCCCTTTCGCCAGCTGGCGTAATAGCGAAGAGGCCCGC
ACCGATCGCCCTTCCCAACAGTTGCGCAGCCTGAATGGCGAATGGGACGCGCCCTGTAGCGGCGCATTAAGCGCGG
CGGGTGTGGTGGTTACGCGCAGCGTGACCGCTACACTTGCCAGCGCCCTAGCGCCCGCTCCTTTCGCTTTCTTCCC
TTCCTTTCTCGCCACGTTCGCCGGCTTTCCCCGTCAAGCTCTAAATCGGGGGCTCCCTTTAGGGTTCCGATTTAGT
GCTTTACGGCACCTCGACCCCAAAAAACTTGATTAGGGTGATGGTTCACGTAGTGGGCCATCGCCCTGATAGACGG
TTTTTCGCCCTTTGACGTTGGAGTCCACGTTCTTTAATAGTGGACTCTTGTTCCAAACTGGAACAACACTCAACCC
TATCTCGGTCTATTCTTTTGATTTATAAGGGATTTTGCCGATTTCGGCCTATTGGTTAAAAAATGAGCTGATTTAA
CAAAAATTTAACGCGAATTTTAACAAAATCATGTGAGCAAAAGGCCAGCAAAAGGCCAGGAACCGTAAAAAGGCCG
CGTTGCTGGCGTTTTTCCATAGGCTCCGCCCCCCTGACGAGCATCACAAAAATCGACGCTCAAGTCAGAGGTGGCG
AAACCCGACAGGACTATAAAGATACCAGGCGTTTCCCCCTGGAAGCTCCCTCGTGCGCTCTCCTGTTCCGACCCTG
CCGCTTACCGGATACCTGTCCGCCTTTCTCCCTTCGGGAAGCGTGGCGCTTTCTCATAGCTCACGCTGTAGGTATC
TCAGTTCGGTGTAGGTCGTTCGCTCCAAGCTGGGCTGTGTGCACGAACCCCCCGTTCAGCCCGACCGCTGCGCCTT
ATCCGGTAACTATCGTCTTGAGTCCAACCCGGTAAGACACGACTTATCGCCACTGGCAGCAGCCACTGGTAACAGG
ATTAGCAGAGCGAGGTATGTAGGCGGTGCTACAGAGTTCTTGAAGTGGTGGCCTAACTACGGCTACACTAGAAGAA
CAGTATTTGGTATCTGCGCTCTGCTGAAGCCAGTTACCTTCGGAAAAAGAGTTGGTAGCTCTTGATCCGGCAAACA
AACCACCGCTGGTAGCGGTGGTTTTTTTGTTTGCAAGCAGCAGATTACGCGCAGAAAAAAAGGATCTCAAGAAGAT
CCTTTGATCTTTTCTACGGGGTCTGACGCTCAGTGGAACGAAAACTCACGTTAAGGGATTTTGGTCATGAGATTAT
CAAAAAGGATCTTCACCTAGATCCTTTTGATCCTCCGGCGTTCAGCCTGTGCCACAGCCGACAGGATGGTGACCAC
CATTTGCCCCATATCACCGTCGGTACTGATCCCGTCGTCAATAAACCGAACCGCTACACCCTGAGCATCAAACTCT
TTTATCAGTTGGATCATGTCGGCGGTGTCGCGGCCAAGACGGTCGAGCTTCTTCACCAGAATGACATCACCTTCCT
CCACCTTCATCCTCAGCAAATCCAGCCCTTCCCGATCTGTTGAACTGCCGGATGCCTTGTCGGTAAAGATGCGGTT
AGCTTTTACCCCTGCATCTTTGAGCGCTGAGGTCTGCCTCGTGAAGAAGGTGTTGCTGACTCATACCAGGCCTGAA
TCGCCCCATCATCCAGCCAGAAAGTGAGGGAGCCACGGTTGATGAGAGCTTTGTTGTAGGTGGACCAGTTGGTGAT
TTTGAACTTTTGCTTTGCCACGGAACGGTCTGCGTTGTCGGGAAGATGCGTGATCTGATCCTTCAACTCAGCAAAA
GTTCGATTTATTCAACAAAGCCGCCGTCCCGTCAAGTCAGCGTAATGCTCTGCCAGTGTTACAACCAATTAACCAA
TTCTGATTAGAAAAACTCATCGAGCATCAAATGAAACTGCAATTTATTCATATCAGGATTATCAATACCATATTTT
TGAAAAAGCCGTTTCTGTAATGAAGGAGAAAACTCACCGAGGCAGTTCCATAGGATGGCAAGATCCTGGTATCGGT
CTGCGATTCCGACTCGTCCAACATCAATACAACCTATTAATTTCCCCTCGTCAAAAATAAGGTTATCAAGTGAGAA
ATCACCATGAGTGACGACTGAATCCGGTGAGAATGGCAAAAGCTTATGCATTTCTTTCCAGACTTGTTCAACAGGC
CAGCCATTACGCTCGTCATCAAAATCACTCGCATCAACCAAACCGTTATTCATTCGTGATTGCGCCTGAGCGAGAC
GAAATACGCGATCGCTGTTAAAAGGACAATTACAAACAGGAATCGAATGCAACCGGCGCAGGAACACTGCCAGCGC
ATCAACAATATTTTCACCTGAATCAGGATATTCTTCTAATACCTGGAATGCTGTTTTCCCGGGGATCGCAGTGGTG
AGTAACCATGCATCATCAGGAGTACGGATAAAATGCTTGATGGTCGGAAGAGGCATAAATTCCGTCAGCCAGTTTA
GTCTGACCATCTCATCTGTAACATCATTGGCAACGCTACCTTTGCCATGTTTCAGAAACAACTCTGGCGCATCGGG
CTTCCCATACAATCGATAGATTGTCGCACCTGATTGCCCGACATTATCGCGAGCCCATTTATACCCATATAAATCA
GCATCCATGTTGGAATTTAATCGCGGCCTCGAGCAAGACGTTTCCCGTTGAATATGGCTCATAACACCCCTTGTAT
TACTGTTTATGTAAGCAGACAGTTTTATTGTTCATGATGATATATTTTTATCTTGTGCAATGTAACATCAGAGATT
TTGAGACACCATGTTCTTTCCTGCGTTATCCCCTGATTCTGTGGATAACCGTATTACCGCCTTTGAGTGAGCTGAT
ACCGCTCGCCGCAGCCGAACGACCGAGCGCAGCGAGTCAGTGAGCGAGGAAGCGGAAGAGCGCCCAATACGCAAAC
CGCCTCTCCCCGCGCGTTGGCCGATTCATTAATGCAGCTGGCACGACAGGTTTCCCGACTGGAAAGCGGGCAGTGA
GCGCAACGCAATTAATGTGAGTTAGCTCACTCATTAGGCACCCCAGGCTTTACACTTTATGCTTCCGGCTCGTATG
TTGTGTGGAATTGTGAGCGGATAACAATTTCACACAGGAAACAGCTATGACCATGATTACGCCAGATTTAATTAAG
GCCTTAATTAGG
Legend
5′ ITR: nt 1-130
3′ ITR: nt 3085-3214
Human thyroxine-binding globulin (TBG) promoter: nt 220-917 (of
which AMBP enhancer (alpha-1-microglobulin/bikunin precursor)
gene transcription regulatory region,: nt 220-426)
Intron: nt 950-1081
Kozak sequence: nt 1089-1102
hARSB Coding sequence: nt 1103-2704
Bovine Growth Hormone (BGH) polyA: nt 2744-2951
Vector backbone nt 3215-6472
SEQ ID No. 9 pAAV2.1.TBG-eGFP
AGCGCCCAATACGCAAACCGCCTCTCCCCGCGCGTTGGCCGATTCATTAATGCAGCTGGCACGACAGGTTTCCCGA
CTGGAAAGCGGGCAGTGAGCGCAACGCAATTAATGTGAGTTAGCTCACTCATTAGGCACCCCAGGCTTTACACTTT
ATGCTTCCGGCTCGTATGTTGTGTGGAATTGTGAGCGGATAACAATTTCACACAGGAAACAGCTATGACCATGATT
ACGCCAGATTTAATTAAGGCTGCGCGCTCGCTCGCTCACTGAGGCCGCCCGGGCAAAGCCCGGGCGTCGGGCGACC
TTTGGTCGCCCGGCCTCAGTGAGCGAGCGAGCGCGCAGAGAGGGAGTGGCCAACTCCATCACTAGGGGTTCCTTGT
AGTTAATGATTAACCCGCCATGCTACTTATCTACGTAGCCATGCTCTAGGAAGATCGGAATTCGCCCTTAAGCTAG
CAGGTTAATTTTTAAAAAGCAGTCAAAAGTCCAAGTGGCCCTTGGCAGCATTTACTCTCTCTGTTTGCTCTGGTTA
ATAATCTCAGGAGCACAAACATTCCAGATCCAGGTTAATTTTTAAAAAGCAGTCAAAAGTCCAAGTGGCCCTTGGC
AGCATTTACTCTCTCTGTTTGCTCTGGTTAATAATCTCAGGAGCACAAACATTCCAGATCCGGCGCGCCAGGGCTG
GAAGCTACCTTTGACATCATTTCCTCTGCGAATGCATGTATAATTTCTACAGAACCTATTAGAAAGGATCACCCAG
CCTCTGCTTTTGTACAACTTTCCCTTAAAAAACTGCCAATTCCACTGCTGTTTGGCCCAATAGTGAGAACTTTTTC
CTGCTGCCTCTTGGTGCTTTTGCCTATGGCCCCTATTCTGCCTGCTGAAGACACTCTTGCCAGCATGGACTTAAAC
CCCTCCAGCTCTGACAATCCTCTTTCTCTTTTGTTTTACATGAAGGGTCTGGCAGCCAAAGCAATCACTCAAAGTT
CAAACCTTATCATTTTTTGCTTTGTTCCTCTTGGCCTTGGTTTTGTACATCAGCTTTGAAAATACCATCCCAGGGT
TAATGCTGGGGTTAATTTATAACTAAGAGTGCTCTAGTTTTGCAATACAGGACATGCTATAAAAATGGAAAGATGT
TGCTTTCTGAGAGACTGCAGAAGTTGGTCGTGAGGCACTGGGCAGGTAAGTATCAAGGTTACAAGACAGGTTTAAG
GAGACCAATAGAAACTGGGCTTGTCGAGACAGAGAAGACTCTTGCGTTTCTGATAGGCACCTATTGGTCTTACTGA
CATCCACTTTGCCTTTCTCTCCACAGGTGTCCAGGCGGCCGCCATGGTGAGCAAGGGCGAGGAGCTGTTCACCGGG
GTGGTGCCCATCCTGGTCGAGCTGGACGGCGACGTAAACGGCCACAAGTTCAGCGTGTCCGGCGAGGGCGAGGGCG
ATGCCACCTACGGCAAGCTGACCCTGAAGTTCATCTGCACCACCGGCAAGCTGCCCGTGCCCTGGCCCACCCTCGT
GACCACCCTGACCTACGGCGTGCAGTGCTTCAGCCGCTACCCCGACCACATGAAGCAGCACGACTTCTTCAAGTCC
GCCATGCCCGAAGGCTACGTCCAGGAGCGCACCATCTTCTTCAAGGACGACGGCAACTACAAGACCCGCGCCGAGG
TGAAGTTCGAGGGCGACACCCTGGTGAACCGCATCGAGCTGAAGGGCATCGACTTCAAGGAGGACGGCAACATCCT
GGGGCACAAGCTGGAGTACAACTACAACAGCCACAACGTCTATATCATGGCCGACAAGCAGAAGAACGGCATCAAG
GTGAACTTCAAGATCCGCCACAACATCGAGGACGGCAGCGTGCAGCTCGCCGACCACTACCAGCAGAACACCCCCA
TCGGCGACGGCCCCGTGCTGCTGCCCGACAACCACTACCTGAGCACCCAGTCCGCCCTGAGCAAAGACCCCAACGA
GAAGCGCGATCACATGGTCCTGCTGGAGTTCGTGACCGCCGCCGGGATCACTCTCGGCATGGACGAGCTGTACAAG
TAATAAGCTTGGATCCAATCAACCTCTGGATTACAAAATTTGTGAAAGATTGACTGGTATTCTTAACTATGTTGCT
CCTTTTACGCTATGTGGATACGCTGCTTTAATGCCTTTGTATCATGCTATTGCTTCCCGTATGGCTTTCATTTTCT
CCTCCTTGTATAAATCCTGGTTGCTGTCTCTTTATGAGGAGTTGTGGCCCGTTGTCAGGCAACGTGGCGTGGTGTG
CACTGTGTTTGCTGACGCAACCCCCACTGGTTGGGGCATTGCCACCACCTGTCAGCTCCTTTCCGGGACTTTCGCT
TTCCCCCTCCCTATTGCCACGGCGGAACTCATCGCCGCCTGCCTTGCCCGCTGCTGGACAGGGGCTCGGCTGTTGG
GCACTGACAATTCCGTGGTGTTGTCGGGGAAGCTGACGTCCTTTCCATGGCTGCTCGCCTGTGTTGCCACCTGGAT
TCTGCGCGGGACGTCCTTCTGCTACGTCCCTTCGGCCCTCAATCCAGCGGACCTTCCTTCCCGCGGCCTGCTGCCG
GCTCTGCGGCCTCTTCCGCGTCTTCGAGATCTGCCTCGACTGTGCCTTCTAGTTGCCAGCCATCTGTTGTTTGCCC
CTCCCCCGTGCCTTCCTTGACCCTGGAAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGACAGCAAGGGGGAGGATTGGGAAGACA
ATAGCAGGCATGCTGGGGACTCGAGTTAAGGGCGAATTCCCGATTAGGATCTTCCTAGAGCATGGCTACGTAGATA
AGTAGCATGGCGGGTTAATCATTAACTACAAGGAACCCCTAGTGATGGAGTTGGCCACTCCCTCTCTGCGCGCTCG
CTCGCTCACTGAGGCCGGGCGACCAAAGGTCGCCCGACGCCCGGGCTTTGCCCGGGCGGCCTCAGTGAGCGAGCGA
GCGCGCAGCCTTAATTAACCTAATTCACTGGCCGTCGTTTTACAACGTCGTGACTGGGAAAACCCTGGCGTTACCC
AACTTAATCGCCTTGCAGCACATCCCCCTTTCGCCAGCTGGCGTAATAGCGAAGAGGCCCGCACCGATCGCCCTTC
CCAACAGTTGCGCAGCCTGAATGGCGAATGGGACGCGCCCTGTAGCGGCGCATTAAGCGCGGCGGGTGTGGTGGTT
ACGCGCAGCGTGACCGCTACACTTGCCAGCGCCCTAGCGCCCGCTCCTTTCGCTTTCTTCCCTTCCTTTCTCGCCA
CGTTCGCCGGCTTTCCCCGTCAAGCTCTAAATCGGGGGCTCCCTTTAGGGTTCCGATTTAGTGCTTTACGGCACCT
CGACCCCAAAAAACTTGATTAGGGTGATGGTTCACGTAGTGGGCCATCGCCCCGATAGACGGTTTTTCGCCCTTTG
ACGCTGGAGTTCACGTTCCTCAATAGTGGACTCTTGTTCCAAACTGGAACAACACTCAACCCTATCTCGGTCTATT
CTTTTGATTTATAAGGGATTTTTCCGATTTCGGCCTATTGGTTAAAAAATGAGCTGATTTAACAAAAATTTAACGC
GAATTTTAACAAAATATTAACGTTTATAATTTCAGGTGGCATCTTTCGGGGAAATGTGCGCGGAACCCCTATTTGT
TTATTTTTCTAAATACATTCAAATATGTATCCGCTCATGAGACAATAACCCTGATAAATGCTTCAATAATATTGAA
AAAGGAAGAGTATGAGTATTCAACATTTCCGTGTCGCCCTTATTCCCTTTTTTGCGGCATTTTGCCTTCCTGTTTT
TGCTCACCCAGAAACGCTGGTGAAAGTAAAAGATGCTGAAGATCAGTTGGGTGCACGAGTGGGTTACATCGAACTG
GATCTCAATAGTGGTAAGATCCTTGAGAGTTTTCGCCCCGAAGAACGTTTTCCAATGATGAGCACTTTTAAAGTTC
TGCTATGTGGCGCGGTATTATCCCGTATTGACGCCGGGCAAGAGCAACTCGGTCGCCGCATACACTATTCTCAGAA
TGACTTGGTTGAGTACTCACCAGTCACAGAAAAGCATCTTACGGATGGCATGACAGTAAGAGAATTATGCAGTGCT
GCCATAACCATGAGTGATAACACTGCGGCCAACTTACTTCTGACAACGATCGGAGGACCGAAGGAGCTAACCGCTT
TTTTGCACAACATGGGGGATCATGTAACTCGCCTTGATCGTTGGGAACCGGAGCTGAATGAAGCCATACCAAACGA
CGAGCGTGACACCACGATGCCTGTAGTAATGGTAACAACGTTGCGCAAACTATTAACTGGCGAACTACTTACTCTA
GCTTCCCGGCAACAATTAATAGACTGGATGGAGGCGGATAAAGTTGCAGGACCACTTCTGCGCTCGGCCCTTCCGG
CTGGCTGGTTTATTGCTGATAAATCTGGAGCCGGTGAGCGTGGGTCTCGCGGTATCATTGCAGCACTGGGGCCAGA
TGGTAAGCCCTCCCGTATCGTAGTTATCTACACGACGGGGAGTCAGGCAACTATGGATGAACGAAATAGACAGATC
GCTGAGATAGGTGCCTCACTGATTAAGCATTGGTAACTGTCAGACCAAGTTTACTCATATATACTTTAGATTGATT
TAAAACTTCATTTTTAATTTAAAAGGATCTAGGTGAAGATCCTTTTTGATAATCTCATGACCAAAATCCCTTAACG
TGAGTTTTCGTTCCACTGAGCGTCAGACCCCGTAGAAAAGATCAAAGGATCTTCTTGAGATCCTTTTTTTCTGCGC
GTAATCTGCTGCTTGCAAACAAAAAAACCACCGCTACCAGCGGTGGTTTGTTTGCCGGATCAAGAGCTACCAACTC
TTTTTCCGAAGGTAACTGGCTTCAGCAGAGCGCAGATACCAAATACTGTCCTTCTAGTGTAGCCGTAGTTAGGCCA
CCACTTCAAGAACTCTGTAGCACCGCCTACATACCTCGCTCTGCTAATCCTGTTACCAGTGGCTGCTGCCAGTGGC
GATAAGTCGTGTCTTACCGGGTTGGACTCAAGACGATAGTTACCGGATAAGGCGCAGCGGTCGGGCTGAACGGGGG
GTTCGTGCACACAGCCCAGCTTGGAGCGAACGACCTACACCGAACTGAGATACCTACAGCGTGAGCTATGAGAAAG
CGCCACGCTTCCCGAAGGGAGAAAGGCGGACAGGTATCCGGTAAGCGGCAGGGTCGGAACAGGAGAGCGCACGAGG
GAGCTTCCAGGGGGAAACGCCTGGTATCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCGGCCTTTTTACGGTTCCTGGCCTTTTG
CTGCGGTTTTGCTCACATGTTCTTTCCTGCGTTATCCCCTGATTCTGTGGATAACCGTATTACCGCCTTTGAGTGA
GCTGATACCGCTCGCCGCAGCCGAACGACCGAGCGCAGCGAGTCAGTGAGCGAGGAAGCGGAAG
Legend
5′ ITR: nt 248-377
3′ UTR: nt 2919-3048
Coding sequence eGFP: nt 1336-2052
TBG promoter: nt 678-1154
Vecor backbone: nt 3052-5612
PROMOTERS
SEQ ID No. 10 TBG promoter
AGGGCTGGAAGCTACCTTTGACATCATTTCCTCTGCGAATGCATGTATAATTTCTACAGAACCTATTAGAAAGGAT
CACCCAGCCTCTGCTTTTGTACAACTTTCCCTTAAAAAACTGCCAATTCCACTGCTGTTTGGCCCAATAGTGAGAA
CTTTTTCCTGCTGCCTCTTGGTGCTTTTGCCTATGGCCCCTATTCTGCCTGCTGAAGACACTCTTGCCAGCATGGA
CTTAAACCCCTCCAGCTCTGACAATCCTCTTTCTCTTTTGTTTTACATGAAGGGTCTGGCAGCCAAAGCAATCACT
CAAAGTTCAAACCTTATCATTTTTTGCTTTGTTCCTCTTGGCCTTGGTTTTGTACATCAGCTTTGAAAATACCATC
CCAGGGTTAATGCTGGGGTTAATTTATAACTAAGAGTGCTCTAGTTTTGCAATACAGGACATGCTATAAAAATGGA
AAGATGTTGCTTTCTGAGAGA
SEQ ID No. 11 alfa-1-antitripsin promoter
GATCTTGCTACCAGTGGAACAGCCACTAAGGATTCTGCAGTGAGAGCAGAGGGCCAGCTAAGTGGTACTCTCCCAG
AGACTGTCTGACTCACGCCACCCCCTCCACCTTGGACACAGGACGCTGTGGTTTCTGAGCCAGGTACAATGACTCC
TTTCGGTAAGTGCAGTGGAAGCTGTACACTGCCCAGGCAAAGCGTCCGGGCAGCGTAGGCGGGCGACTCAGATCCC
AGCCAGTGGACTTAGCCCCTGTTTGCTCCTCCGATAACTGGGGTGACCTTGGTTAATATTCACCAGCAGCCTCCCC
CGTTGCCCCTCTGGATCCACTGCTTAAATACGGACGAGGACAGGGCCCTGTCTCCTCAGCTTCAGGCACCACCACT
GACCTGGGACAGT
SEQ ID No. 12 albumin
GCATGCTTCCATGCCAAGGCCCACACTGAAATGCTCAAATGGGAGACAAAGAGATTAAGCTCTTATGTAAAATTTG
CTGTTTTACATAACTTTAATGAATGGACAAAGTCTTGTGCATGGGGGTGGGGGTGGGGTTAGAGGGGAACAGCTCC
AGATGGCAAACATACGCAAGGGATTTAGTCAAACAACT
TTTTGGCAAAGATGGTATGATTTTGTAATGGGGTAGGAACCAATGAAATGCGAGGTAAGTATGGTTAATGATCTAC
AGTTATTGGTTAAAGAAGTATATTAGAGCGAGTCTTTCTGCACACAGATCACCTTTCCTATCAACCCC
SEQ ID No.13 phosphoglycerate kinase promoter
CACGGGGTTGGGGTTGCGCCTTTTCCAAGGCAGCCCTGGGTTTGCGCAGGGACGCGGCTGCTCTGGGCGTGGTTCC
GGGAAACGCAGCGGCGCCGACCCTGGGTCTCGCACATTCTTCACGTCCGTTCGCAGCGTCACCCGGATCTTCGCCG
CTACCCTTGTGGGCCCCCCGGCGACGCTTCCTGCTCCGCCCCTAAGTCGGGAAGGTTCCTTGCGGTTCGCGGCGTG
CCGGACGTGACAAACGGAAGCCGCACGTCTCACTAGTACCCTCGCAGACGGACAGCGCCAGGGAGCAATGGCAGCG
CGCCGACCGCGATGGGCTGTGGCCAATAGCGGCTGCTCAGCGGGGCGCGCCGAGAGCAGCGGCCGGGAAGGGGCGG
TGCGGGAGGCGGGGTGTGGGGCGGTAGTGTGGGCCCTGTTCCTGCCCGCGCGGTGTTCCGCATTCTGCAAGCCTCC
GGAGCGCACGTCGGCAGTCGGCTCCCTCGTTGACCGAATCACCGACCTCTCTCCCCAGGG
SEQ ID No. 14 CMV promoter
TAGTTATTAATAGTAATCAATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAATGACGTATGTTCCCATAGTAA
CGCCAATAGGGACTTTCCATTGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGTGATGCGGTTTTGGC
AGTACATCAATGGGCGTGGATAGCGGTTTGACTCACGGGGATTTCCAAGTCTCCACCCCATTGACGTCAATGGGAG
TTTGTTTTGGCACCAAAATCAACGGGACTTTCCAAAATGTCGTAACAACTCCGCCCCATTGACGCAAATGGGCGGT
AGGCGTGTACGGTGGGAGGTCTATATAAGCAGAGCTGGTTTAGTGAACCGT

### Materials and Methods

### Animal Colony

MPS VI mice were maintained at the Cardarelli Hospital Animal House (Naples, Italy). Animals were raised in accordance with the Institutional Animal Care and Use Committee (IACUC) guidelines for the care and use of animals in research. This mouse model carries a targeted disruption of the ARSB locus⁶⁴ and is made immune-tolerant to human ARSB by transgenic insertion of the C91S hARSB mutant, resulting in the production of inactive hARSB⁶⁵. Six out of 38 MPS VI mice from the same genetic background had the C91S hARSB transgene inserted into the ROSA26 locus⁶⁶, while the remaining presented random integrations of this transgene¹⁵. Genotype analysis was performed by polymerase chain reaction (PCR) on genomic DNA obtained from the tail, as previously described 15.

### Plasmid and Vector Production

The plasmid pAAV2.1.TBG-hARSB (see FIG. 7) encoding the hARSB protein was generated, as described previously¹⁹. The gene therapy AAV2/8.TBG.hARSB and the control AAV2/8.TBG.eGFP vectors were produced by the AAV Vector Core (Telethon Institute of Genetics and Medicine [TIGEM], Pozzuoli, Naples, Italy), as previously described¹⁴.

### Treatment Administration

MPS VI mice were treated with gene therapy and/or ERT through intravenous retro-orbital injections, starting from p30 to avoid vector dilution due to hepatocyte proliferation^{13, 56, 57} and were followed up to 6-7 months (180-210 post-natal days) of age. MPS VI mice were treated with a single injection of the AAV2/8.TBG.hARSB vector at either 2×10¹¹ or 6×10¹¹ gc/kg and/or monthly injections of 1 mg/kg rhARSB protein (Naglazyme, BioMarin Europe, London, UK), appropriately diluted in phosphate buffered saline (PBS). As control, MPS VI mice either received monthly administrations of PBS (n=1) or a single injection of the control AAV2/8.TBG.eGFP vector (n=1) or were left untreated (n=8). Both male and female mice are included in each group of treatment.

### Blood, Urine, and Tissue Collection

Blood was collected each month from treated and control mice and before ERT administration in mice receiving ERT with or without gene therapy. Serum samples were collected via eye bleeding and centrifuged at 10,000×g in a microcentrifuge (Z 216 MK, HERMLE) for 10 min at 4° C. to obtain the serum.

Urine was also collected monthly before each ERT administration in mice receiving ERT with or without gene therapy. Specifically, mice were put in metabolic cages for 24 hours. The urine samples were briefly centrifuged to remove debris and stored at −20° C.

Mice were sacrificed 5 or 6 months following the start of treatment and 1 month after the last ERT administration. A cardiac perfusion with PBS was performed, and the liver, kidney, spleen and heart were collected. Tissue samples were fixed in a methacarn solution (30% chloroform, 60% methanol, 10% acetic acid) for 24 h or frozen in dry ice (for ARSB activity and GAG quantitative assays).

### Immune Capture Assay for Determination of Serum ARSB Levels?

Serum ARSB levels were measured by an immune capture assay based on the use of a specific anti-hARSB polyclonal antibody (Covalab, Villeurbanne, France). Ninety-six-well plates (Nunclon, Roskilde, Denmark) were coated with 5 μg/ml in 0.1 M NaHCO₃(100 μl/well) and incubated overnight (0/N) at 4° C. The following day, plates were washed twice with 0.25 M NaCl/0.02 M Tris, pH 7, and then blocked with 1% milk in 0.25 M NaCl/0.02 M Tris, pH 7 (blocking solution), for 2 h at room temperature. Plates were washed again, as described above, and then 50 μl of standard and unknown samples (diluted 1:10) were added to each well. Plates were shaken for 1 h at room temperature and then incubated at 4° C. O/N. The following day, plates were shaken for 1 h at room temperature and then washed 2× with 0.25 M NaCl/0.02 M Tris, pH 7. In total, 100 μl of 5 mM 4-methylumbelliferylsulfate potassium salt (4-MUS, Sigma-Aldrich, Milan, Italy) substrate was added to each well and then incubated at 37° C. for 4 h. The reaction was stopped by the addition of 100 μl/well of stop solution (glycine 0.2 M). Plates were shaken for 10 min at room temperature and the fluorescence was read (excitation 365 nm/emission 460 nm) on a multiplate fluorimeter (TECAN Infinite F200, Männedorf, Switzerland). Serum ARSB was determined based on a rhARSB (Naglazyme, BioMarin Europe, London, UK) standard curve and expressed as pg/mL.

### AAV Vector Genome Distribution

Genomic DNA was extracted from the livers using the DNeasy Blood and Tissue Extraction kit (Qiagen). Real-time PCR analysis was performed on 100 ng of genomic DNA using a set of primers/probe (Fw 5′-TCTAGTTGCCAGCCATCTGTTGT-3′ (SEQ ID NO. 15), Rev 5′-TGGGAGTGGCACCTTCCA-3′ (SEQ ID NO. 16), Probe 5′-TCCCCCGTGCCTTCCTTGACC-3′ (SEQ ID NO. 17)) specific for the viral genome and Taq-Man universal PCR master mix (Applied Biosystems, Foster City, Calif., USA). Amplification was run on a 7300 Real-Time PCR system (Applied Biosystems) with standard cycles. All the reactions were performed in triplicate.

### Assay for ARSB and GASB Enzymatic Activity Evaluation in Tissues

Tissues, i.e liver, kidney and spleen, were homogenized in water and protein concentrations were determined with the bicinchoninic acid (BCA) protein assay kit (Pierce Protein Research Products, Thermo Fisher Scientific, Rockford, Ill., USA). The ARSB assay was performed, as previously described⁶⁷. Briefly, 30 μg of protein was incubated with 40 μl of the fluorogenic4-methylumbelliferyl sulfate substrate (12.5 mM; Sigma-Aldrich, Saint Louis, Mo., USA) for 3 h at 37° C. in the presence of 40 μl silver nitrate (0.75 mM; Carlo Erba, Milan, Italy), which is known to inhibit the activity of other sulfatases. The reaction was stopped by adding 200 μl of carbonate glycine stop buffer and the fluorescence of the 4-methylumbelliferone liberated was measured on a multiplate reader (TECAN Infinite F200) at 365 nm (excitation) and 460 nm (emission).

β-glucuronidase (GUSB) assay was performed, as previously described⁶⁸. Briefly, 200 μg of protein was incubated with 400 μl of GUS assay buffer (50 mM NaPO₄ pH 7, 5 mM DTT, 1 mM EDTA, 0.1% Triton X-100) and 100 μl of the fluorogenic 4-methylumbelliferyl-β-d-glucuronide (MUG) substrate (5 mM; Sigma-Aldrich, Saint Louis, Mo., USA) for 30 min at 37° C. The reaction was stopped by adding 900 μl of stop buffer (0.2 mM Na₂CO₃, pH 9.5) to 50 μl of sample. The fluorescence was measured on a multiplate reader (GloMax-Multi detection system Promega) at 388 nm (excitation) and 480 nm (emission).

Enzyme activities were calculated with a standard curve of the fluorogenic4-methylumbelliferone product (12.5 mM; Sigma-Aldrich, Saint Louis, Mo., USA). For tissue lysates the activity was expressed as nanomoles per milligram of protein per hour (nmol/mg/h).

### Quantitative Analyses of GAG Accumulation in Tissues and Urine

Urine samples were diluted 1:50 in water to measure GAG content. One hundred microliters of diluted urine or 250 μg of protein extract from the liver, spleen, and kidney was used for the GAG assay, as previously described⁶⁹. GAG concentrations were determined on the basis of a dermatan sulfate standard curve (Sigma-Aldrich, Saint Louis, Mo., USA). Tissue GAGs were expressed as micrograms of GAG per milligram of protein (μg GAG/mg protein). Urinary GAGs were normalized to creatinine content which was measured with a creatinine assay kit (Quidel, San Diego, Calif., USA). Thus, the units of urinary GAGs are micrograms per micromole of creatinine (μg GAG/μmol creatinine). Urinary GAGs were reported as percentage of AF control mice. The urinary GAG levels measured at each time point were averaged for each group.

### Alcian Blue Staining

After methacarn fixation, all the tissues (liver, spleen, kidney, heart) were dehydrated through immersion in alcohols at increasing concentration (70%, 80%, 90%, 100%) and then in xylene. Tissues were embedded in paraffin and sectioned into 7-μm-thick serial sections on a microtome. Tissue sections were de-paraffinized, rehydrated through immersion in alcohols at decreasing concentration (100%, 95%, 80% and 70%) and then water and stained with 1% Alcian blue (Sigma-Aldrich, Saint Louis, Mo., USA) in hydrochloric acid for 10 seconds. Counterstaining was performed for 1 min with nuclear-fast red (Sigma-Aldrich, Saint Louis, Mo., USA). Alcian blue staining in heart valves and myocardium was quantified to provide a measure of GAG storage. Specifically, Alcian blue staining was quantified by measuring RGB intensity on histological section using the Image J software. RGB may assume integer values from 0 to 255. The more intense is the Alcian Blue staining, the lower is the RBG value. Four different areas were randomly selected in each valve. As far as myocardium, five areas corresponding to Alcian blue spots were randomly selected per each section. Where Alcian blue spots were not present, as in NR and some treated mice, five equivalent areas were randomly selected. RGB was measured per each area and then averaged for each animal and each group of animals, as reported in FIGS. 4 and 5.

### GAG Levels

### Sample Preparations

### Urine

Keep urine frozen (−20/−80). Before use, spin urines at 200 g for 2 minutes to remove debris and collect supernatant. Urine samples should be diluted between 1:50 in water. Depending on the assay used, use 50 ul of sample (microplate assay) or 100 ul sample (cuvette assay).

### Tissue Lysates

Homogenize tissue in the tissue lyser (QIAGEN) in water with LAP (protease inhibitor cocktail tablets) (Roche), Pellet cell debris (14000 rpr, 15-20 min, 4° C.), Collect supernatant, Measure protein concentration, Dilute tissue at 5 ug/ul.

250 ug is loaded in a final volume of 100 ul (cuvette assay); 125 ug is loaded in a final volume of 50 ul (multiplate). (Please note that the amount of protein to be tested depends on the concentration of the sample and the expected GAG concentration).

### Materials

Dermatan Sulfate stock (2 mg/ml) also known as chondroitin sulphate B (Sigma C3788); Dimethylmethylene Blue (DMB) Reagent (10.7 mg DMB in 55 mM formate buffer, pH 3.3);

2M Tris (base);

F96 Maxisorp Nunc-immuno plate (Nalge Nunc part n. 442404) for multiplate protocol.

For cuvette assay:

Plastic cuvettes;

90% formic acid;

1,9-Dimethyl-methylene blue (DMB; Sigma-Aldrich 341088);

Tris base (MW 121,14);

Tissue culture filter 0.22 um PES

### Preparation of Solutions

### Dmb Formate:

1. mix 897.2 ml H₂O with 2.8 ml of 90% formic acid in a 1 L cylinder
2. measure pH of the solution while stirring. Add NaOH to bring the solution to a pH of 3.29
3. Bring the solution to 1 L with H2O. This will increase pH to 3.33
4. Wrap a 1 L sterilized glass bottle with aluminium to protect from light.
5. Add about 500 ml of Formate buffer in the bottle
6. Slowly add 10.7 mg (0.0107 g) of DMB to the Formate buffer during 2 minutes while stirring
7. Continue to stir for about 1 hour and then add the other 500 ml of formate buffer
8. Stir the solution for about 8 hours to overnight

### 2M Tris

Weight 60.57 g of tris and add about 200 ml water
Dissolve Tris by stirring (if needed can heat the solution to allow dissolution)
Bring to 250 ml with water
Filter the solution with 0.2 um filters

### Measure pH (11.34)

### Procedure

Prepare the DS standard from the 2 mg/ml stock. The DS should be diluted with water to obtain concentrations of 40, 20, 10, 5, 2.5, and 1.25 ug/ml (standard can be stored at −20° C.)

| STANDARD | DS | Water |
|---|---|---|
| A) 40 ug/ml | 10 ul stock | 490 |
| B) 20 ug/ml | 150 ul standard A | 150 |
| C) 10 ug/ml | 150 ul standard B | 150 |
| D) 5 ug/ml | 150 ul standard C | 150 |
| E) 2.5 ug/ml | 150 ul standard D | 150 |
| F) 1.25 ug/ml | 150 ul standard E | 150 |

### Prepare Dilutions of the Samples (in Water)

If done in multiplate
load in each plate well a blank (water), the standard and the diluted samples in duplicate (50 ul/well)
Calculate the amount of DMB-Tris reagent required for all samples (each sample require 275 ul of reagent). Prepare the DMB-Tris reagent by combining ten parts of the DMB reagent with one part of 2M Tris (Es. 500 ul DMB+50 ul Tris). The resultant solution is only stable for about 15 minutes.
Add 275 ul of DMB-Tris to each well (use multichannel pipette to be fast). Read the plate at 520 nm one minute after adding the DMB-Tris reagent to the last column.

If done in cuvette
prepare as much plastic cuvettes as the number of samples-standard you have (in duplicate)
load in the cuvettes a blank (water), the standard and the diluted samples in duplicate (100 ul/each).
Calculate the amount of DMB-Tris reagent required for all samples (each sample require 550 ul of reagent). Prepare the DMB-Tris reagent by combining ten parts of the DMB reagent with one part of 2M Tris (Es. 500 ul DMB+50 ul Tris). The resultant solution is only stable for about 15 minutes.
Add 550 ul of DMB-Tris to each cuvette (since the reaction is stable for few minutes, we usually add the buffer to 3-5 cuvettes and read absorbance; then we add buffer to additional 3-5 cuvette and so on). Mix cuvette and read absorbance at 520 nm (spectrophotometer).

### Results Analysis

### Tissue Lysates

Calculate GAG concentration of the diluted sample form the standard curve (GAG concentration in ug/ml)
Divide this concentration by the protein concentration of the diluted sample (You will obtain ug GAG/mg protein)

### Urine Samples

Normalize on creatinine concentration: on the same urine dilution used for GAG determination, measure creatinine concentration with the QUIDEL Microvue Creatinine Assay Kit. Divide GAG concentration (ug/ml) for Creatinine concentration (umol/ml) to obtain ug GAG/umol of creatinine.

### Creatinine Assay

Follow Creatinine Assay Kit (Quidel San Diego, Calif.; catalog number 8009) protocol. Dilute standards and controls (from the kit) 1:40.

For samples the same dilution used for GAG measurement should be used. If samples are diluted differently, the different dilution should be kept in mind before normalyzing. Calculate creatinine concentration from standard curve. Add lower point of the creatinine curve if necessary, i.e 2.5 and 1.25 mmol/L.

### Statistical Analyses

All results are expressed as mean±standard error (SE). Statistical comparisons were made using either t-test or one-way analysis of variance (ANOVA); the Tukey post hoc test was used. Statistical significance was considered if p<0.05. The exact p value for each comparison follows:

Table 1. Serum ARSB: The ANOVA p value is 2.00 e⁻¹⁶; the p value of NR vs AF is: 4.88e⁻¹³; the p value of ERT vs AF is: 1.00; the p value of AAV 2×10¹¹ vs AF is: 0.99; the p value of AAV 2×10¹¹+ERT vs AF is: 0.83; the p value of AAV 6×10¹¹ vs AF is: 0.02; the p value of AAV 6×10¹¹+ERT vs AF is: 6.90e⁻⁴;

Liver genome copies: The ANOVA p value is 4.85e⁻⁸; the p value of AAV 2×10¹¹ vs AAV 6×10¹¹ is: 2.20e⁻⁶; the p value of AAV 2×10¹¹+ERT vs AAV 6×10¹¹+ERT is: 3.70e⁻⁶; the p value of AAV 2×10¹¹ vs AAV 2×10¹¹+ERT is: 0.98; the p value of AAV 6×10¹¹ vs AAV 6×10¹¹+ERT is: 0.99;

Liver ARSB activity: The ANOVA p value is 3.75e⁻⁹; the p value of ERT vs AF is: 0.58; the p value of AAV 2×10¹¹ vs AF is: 0.03; the p value of AAV 2×10¹¹+ERT vs AF is: 1.00e⁻³; the p value of AAV 6×10¹¹ vs AF is: 1.00e⁻⁷; the p value of AAV 6×10¹¹+ERT vs AF is: <3.75e⁹; the p value of AAV 2×10¹¹ vs AAV 6×10¹¹ is: 1.00e⁻³; the p value of AAV 2×10¹¹+ERT vs AAV 6×10¹¹+ERT is: 6.00e⁻³; the p value of NR vs AF is 2.17e⁻⁸.

Liver GAGs: The ANOVA p value is 7.93e⁻²¹; the p value of NR vs AF is <7.93e⁻²¹; the p value of ERT vs AF is: <7.93e⁻²¹; the p value of AAV 2×10¹¹ vs AF is: <7.93e⁻²¹; the p value of AAV 2×10¹¹+ERT vs AF is: <7.93e⁻²¹; the p value of AAV 6×10¹¹ vs AF is: <7.93e⁻²¹; the p value of AAV 6×10¹¹+ERT vs AF is: <7.93e⁻²¹; the p value of ERT vs NR is: 0.99; the p value of AAV 2×10¹¹ vs NR is: 0.99; the p value of AAV 2×10¹¹+ERT vs NR is: 0.99; the p value of AAV 6×10¹¹ vs NR is: 0.99; the p value of AAV 6×10¹¹+ERT vs NR is: 0.99;

Kidney ARSB activity: The ANOVA p value is 1.28e⁻⁵; the p value of ERT vs AF is: 0.22; the p value of AAV 2×10¹¹ vs AF is: 2.00e⁻³; the p value of AAV 2×10¹¹+ERT vs AF is: 8.20e⁻⁵; the p value of AAV 6×10¹¹ vs AF is: 8.30e⁻⁵; the p value of AAV 6×10¹¹+ERT vs AF is: 1.00e⁻³; the p value of NR vs AF is 1.80e⁻⁸.

Kidney GAGs: The ANOVA p value is 1.16e⁻¹⁵; the p value of NR vs AF is <1.16e⁻¹⁵; the p value of ERT vs AF is: 1.00e⁻⁷; the p value of AAV 2×10¹¹ vs AF is: 4.00e⁻⁷; the p value of AAV 2×10¹¹+ERT vs AF is: <1.16e⁻¹⁵; the p value of AAV 6×10¹¹ vs AF is: <1.16e⁻¹⁵; the p value of AAV 6×10¹¹+ERT vs AF is: <1.16e⁻¹⁵; the p value of ERT vs NR is: 0.42; the p value of AAV 2×10¹¹ vs NR is: 0.03; the p value of AAV 2×10¹¹+ERT vs NR is: 0.99; the p value of AAV 6×10¹¹ vs NR is: 0.99; the p value of AAV 6×10¹¹+ERT vs NR is: 0.99;

Spleen ARSB activity: The ANOVA p value is 5.73e⁶; the p value of ERT vs AF is: 0.20; the p value of AAV 2×10¹¹ vs AF is: 7.00e⁻³; the p value of AAV 2×10¹¹+ERT vs AF is: 1.12e⁻⁴; the p value of AAV 6×10¹¹ vs AF is: 2.46e⁻⁴; the p value of AAV 6×10¹¹+ERT vs AF is: 2.46e⁻⁵; the p value of NR vs AF is 4.28e⁻⁹.

Spleen GAGs: The ANOVA p value is 1.49e⁻¹⁸; the p value of NR vs AF is <1.49e⁻¹⁸; the p value of ERT vs AF is: 1.08e⁻¹¹; the p value of AAV 2×10¹¹ vs AF is: 1.67e⁻¹⁰; the p value of AAV 2×10¹¹+ERT vs AF is: <1.49e⁻¹⁸; the p value of AAV 6×10¹¹ vs AF is: <1.49e⁻⁸; the p value of AAV 6×10¹¹+ERT vs AF is: <1.49e⁻¹⁸; the p value of ERT vs NR is: 0.66; the p value of AAV 2×10¹¹ vs NR is: 0.04; the p value of AAV 2×10¹¹+ERT vs NR is: 0.98; the p value of AAV 6×10¹¹ vs NR is: 0.99; the p value of AAV 6×10¹¹+ERT vs NR is: 0.99.

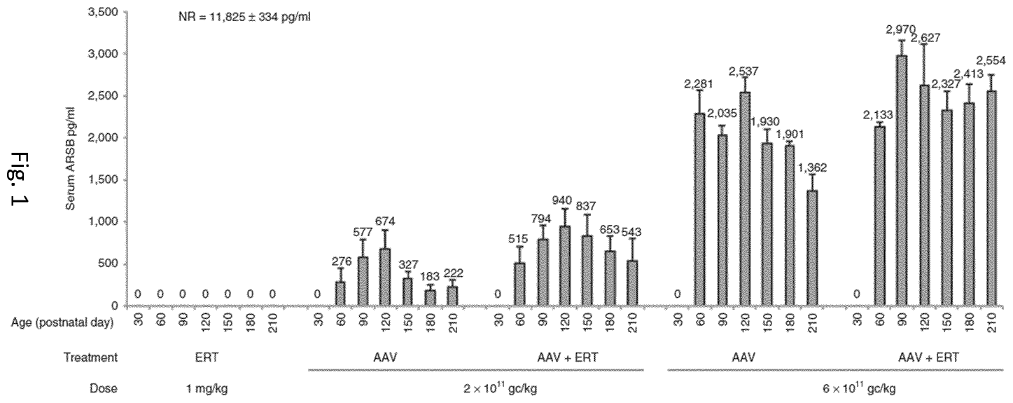

FIG. 1. Post-natal day 30: the ANOVA p value is 6.25e⁻¹²; the p value of NR vs AF is 3.32e⁻⁷; the p value of ERT vs AF is: 1.00; the p value of AAV 2×10¹¹ vs AF is: 1.00; the p value of AAV 2×10¹¹+ERT vs AF is: 1.00; the p value of AAV 6×10¹¹ vs AF is: 1.00; the p value of AAV 6×10¹¹+ERT vs AF is: 1.00; post-natal day 60: the ANOVA p value is 1.09e⁻¹⁴; the p value of NR vs AF is: 7.85e⁻¹²; the p value of ERT vs AF is: 1.00; the p value of AAV 2×10¹¹ vs AF is: 0.99; the p value of AAV 2×10¹¹+ERT vs AF is: 0.99; the p value of AAV 6×10¹¹ vs AF is: 0.73; the p value of AAV 6×10¹¹+ERT vs AF is: 0.72; post-natal day 90: the ANOVA p value is 4.03e⁻¹⁷; the p value of NR vs AF is: <4.03e⁻¹⁷; the p value of ERT vs AF is: 1.00; the p value of AAV 2×10¹¹ vs AF is: 0.99; the p value of AAV 2×10¹¹+ERT vs AF is: 0.99; the p value of AAV 6×10¹¹ vs AF is: 0.91; the p value of AAV 6×10¹¹+ERT vs AF is: 0.56; post-natal day 120: the ANOVA p value is 2.45e⁻¹⁶; the p value of NR vs AF is: <2.45e⁻⁶; the p value of ERT vs AF is: 1.00; the p value of AAV 2×10¹¹ vs AF is: 0.99; the p value of AAV 2×10¹¹+ERT vs AF is: 0.99; the p value of AAV 6×10¹¹ vs AF is: 0.75; the p value of AAV 6×10¹¹+ERT vs AF is: 0.64; post-natal day 150: the ANOVA p value is 5.67e⁻²¹; the p value of NR vs AF is: <5.67e⁻²¹; the p value of ERT vs AF is: 1.00; the p value of AAV 2×10¹¹ vs AF is: 0.99; the p value of AAV 2×10¹¹+ERT vs AF is: 0.99; the p value of AAV 6×10¹¹ vs AF is: 0.78; the p value of AAV 6×10¹¹+ERT vs AF is: 0.51; post-natal day 180: the ANOVA p value is 5.06e⁻²¹; the p value of NR vs AF is: <5.06e⁻²¹; the p value of ERT vs AF is: 1.00; the p value of AAV 2×10¹¹ vs AF is: 0.99; the p value of AAV 2×10¹¹+ERT vs AF is: 0.99; the p value of AAV 6×10¹¹ vs AF is: 0.79; the p value of AAV 6×10¹¹+ERT vs AF is: 0.47; post-natal day 210: the ANOVA p value is 7.75e⁻¹⁵; the p value of NR vs AF is: 5.16e⁻²; the p value of ERT vs AF is: 1.00; the p value of AAV 2×10¹¹ vs AF is: 0.99; the p value of AAV 2×10¹¹+ERT vs AF is: 0.99; the p value of AAV 6×10¹¹ vs AF is: 0.97; the p value of AAV 6×10¹¹+ERT vs AF is: 0.63.

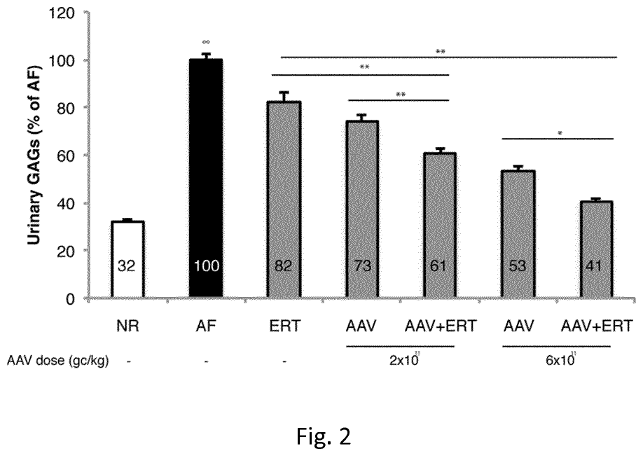

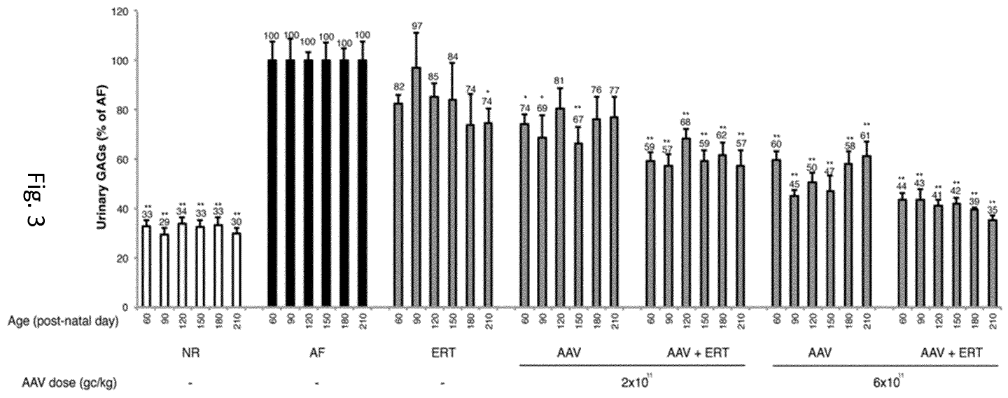

FIG. 2. The ANOVA p value is <2.00 e⁻¹⁶; the p value of NR vs AF is <2.00e⁻¹⁶; the p value of ERT vs AF is: 6.01e⁻⁵; the p value of AAV 2×10¹¹ vs AF is: <2.00e⁻¹⁶; the p value of AAV 2×10¹¹+ERT vs AF is: <2.00e⁻¹⁶; the p value of AAV 6×10¹¹ vs AF is: <2.00e⁻¹⁶; the p value of AAV 6×10¹¹+ERT vs AF is: <2.00e⁻¹⁶; the p value of ERT vs NR is: <2.00e⁻¹⁶; the p value of AAV 2×10¹¹ vs NR is: <2.00e⁻¹⁶; the p value of AAV 2×10¹¹+ERT vs NR is: <2.00e⁻¹⁶; the p value of AAV 6×10¹¹ vs NR is: 4.90e⁻¹°; the p value of AAV 6×10¹¹+ERT vs NR is: 0.08; the p value of AAV 2×10¹¹ vs AAV 6×10¹¹ is: 4.20e⁻⁶; the p value of AAV 2×10¹¹+ERT vs AAV 6×10¹¹+ERT is: 2.86e⁻⁶; the p value of AAV 2×10¹¹+ERT vs ERT is: 3.81e⁻⁷; the p value of AAV 2×10¹¹+ERT vs AAV 2×10¹¹ is: 3.57e⁻³; the p value of AAV 6×10¹¹+ERT vs ERT is: <2.00e⁻¹⁶; the p value of AAV 6×10¹¹+ERT vs AAV 6×10¹¹ is: 0.04.

FIGS. 5 and 8. RGB quantification in heart valves. The ANOVA p value is 4.08e⁻⁴; the p value of NR vs AF is 3.24e⁻⁴; the p value of ERT vs AF is: 0.24; the p value of AAV 2×10¹¹ vs AF is: 0.30; the p value of AAV 2×10¹¹+ERT vs AF is: 0.07; the p value of AAV 6×10¹¹ vs AF is: 9.93e⁻³; the p value of AAV 6×10¹¹+ERT vs AF is: 2.25e⁻³; the p value of ERT vs NR is: 0.04; the p value of AAV 2×10¹¹ vs NR is: 0.01; the p value of AAV 2×10¹¹+ERT vs NR is: 0.05; the p value of AAV 6×10¹¹ vs NR is: 0.62; the p value of AAV 6×10¹¹+ERT vs NR is: 0.95.

FIGS. 5 and 9 RGB quantification in myocardium. The ANOVA p value is 1.16e⁻⁶; the p value of NR vs AF is <1.16e⁻⁶; the p value of ERT vs AF is: 8.38e⁻⁴; the p value of AAV 2×10¹¹ vs AF is: 2.31e⁻³; the p value of AAV 2×10¹¹+ERT vs AF is: 7.37e⁻⁵; the p value of AAV 6×10¹¹ vs AF is: 2.29e⁻⁵; the p value of AAV 6×10¹¹+ERT vs AF is: 3.60e⁻⁶; the p value of ERT vs NR is: 0.02; the p value of AAV 2×10¹¹ vs NR is: 1.48e⁻³; the p value of AAV 2×10¹¹+ERT vs NR is: 0.02; the p value of AAV 6×10¹¹ vs NR is: 0.43; the p value of AAV 6×10¹¹+ERT vs NR is: 0.96.

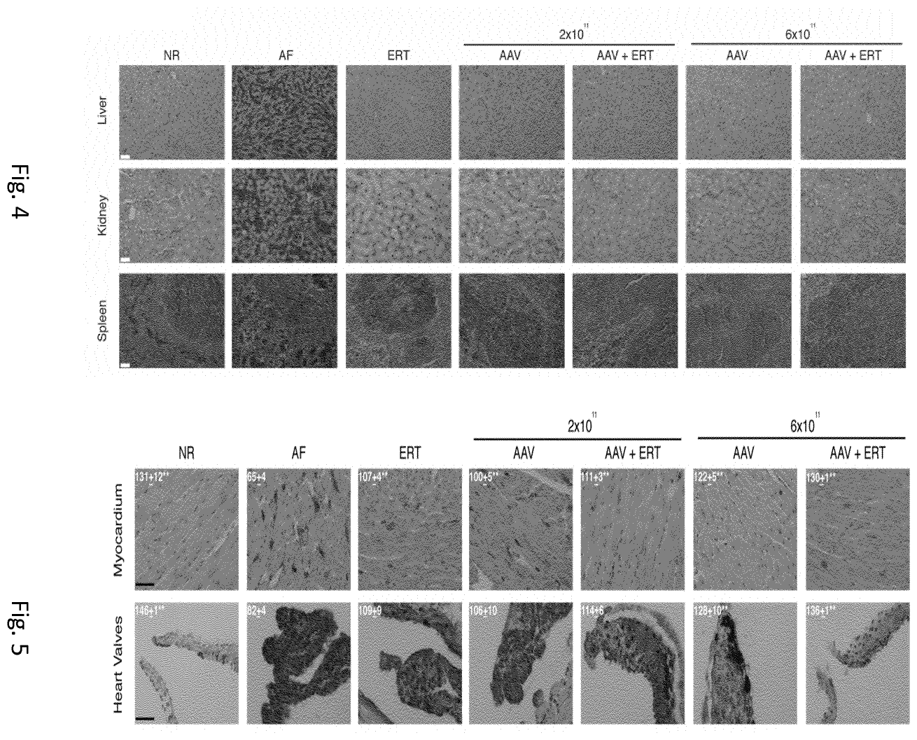

FIG. 3. Post-natal day 60: the ANOVA p value is 5.08e⁻¹⁸; the p value of NR vs AF is: <5.08e⁻¹⁸; the p value of ERT vs AF is: 0.37; the p value of AAV 2×10¹¹ vs AF is: 0.03; the p value of AAV 2×10¹¹+ERT vs AF is: 1.17e⁻⁵; the p value of AAV 6×10¹¹ vs AF is: 1.99e⁻⁴; the p value of AAV 6×10¹¹+ERT vs AF is: 1.11e⁻⁷; the p value of ERT vs NR is: 5.19e⁻⁸; the p value of AAV 2×10¹¹ vs NR is: 7.04e⁻⁷; the p value of AAV 2×10¹¹+ERT vs NR is: 5.51e⁻⁴; the p value of AAV 6×10¹¹ vs NR is: 7.00e⁻³; the p value of AAV 6×10¹¹+ERT vs NR is: 0.76; post-natal day 90: the ANOVA p value is 5.97e⁻¹⁶; the p value of NR vs AF is: <5.97e⁻¹⁶; the p value of ERT vs AF is: 0.99; the p value of AAV 2×10¹¹ vs AF is: 0.02; the p value of AAV 2×10¹¹+ERT vs AF is: 1.37e⁻⁴; the p value of AAV 6×10¹¹ vs AF is: 1.36e⁻⁵; the p value of AAV 6×10¹¹+ERT vs AF is: 2.94e⁻⁶; the p value of ERT vs NR is: 1.63e⁻⁸; the p value of AAV 2×10¹¹ vs NR is: 9.76e⁻⁵; the p value of AAV 2×10¹¹+ERT vs NR is: 3.00e⁻³; the p value of AAV 6×10¹¹ vs NR is: 0.54; the p value of AAV 6×10¹¹+ERT vs NR is: 0.82; post-natal day 120: the ANOVA p value is 1.33e⁻²⁰; the p value of NR vs AF is: 1.46e⁻¹²; the p value of ERT vs AF is: 0.41; the p value of AAV 2×10¹¹ vs AF is: 0.09; the p value of AAV 2×10¹¹+ERT vs AF is: 1.20e⁻⁴; the p value of AAV 6×10¹¹ vs AF is: 1.12e⁻⁷; the p value of AAV 6×10¹¹+ERT vs AF is: 6.22e⁻¹⁰; the p value of ERT vs NR is: 4.18e⁻¹⁰; the p value of AAV 2×10¹¹ vs NR is: 6.96e⁻¹⁰; the p value of AAV 2×10¹¹+ERT vs NR is: 1.55e⁻⁷; the p value of AAV 6×10¹¹ vs NR is: 0.13; the p value of AAV 6×10¹¹+ERT vs NR is: 0.91; post-natal day 150: the ANOVA p value is 9.28e⁻¹⁵; the p value of NR vs AF is: 2.04e⁻¹¹; the p value of ERT vs AF is: 0.64; the p value of AAV 2×10¹¹ vs AF is: 4.00e⁻³; the p value of AAV 2×10¹¹+ERT vs AF is: 4.84e⁻⁵; the p value of AAV 6×10¹¹ vs AF is: 3.47e⁻⁶; the p value of AAV 6×10¹¹+ERT vs AF is: 3.51e⁻⁷; the p value of ERT vs NR is: 2.30e⁻⁶; the p value of AAV 2×10¹¹ vs NR is: 2.6e⁻⁴; the p value of AAV 2×10¹¹+ERT vs NR is: 2.00e⁻³; the p value of AAV 6×10¹¹ vs NR is: 0.51; the p value of AAV 6×10¹¹+ERT vs NR is: 0.90; post-natal day 180: the ANOVA p value is 5.20e⁻¹⁴; the p value of NR vs AF is: 7.37e⁻¹²; the p value of ERT vs AF is: 0.09; the p value of AAV 2×10″ vs AF is: 0.08; the p value of AAV 2×10¹¹+ERT vs AF is: 9.99e⁻⁵; the p value of AAV 6×10″ vs AF is: 2.47e⁻⁴; the p value of AAV 6×10¹¹+ERT vs AF is: 9.80e⁻⁸; the p value of ERT vs NR is: 2.40e⁻⁴; the p value of AAV 2×10¹¹ vs NR is: 2.68e⁻⁶; the p value of AAV 2×10¹¹+ERT vs NR is: 7.73e⁻⁴; the p value of AAV 6×10¹¹ vs NR is: 0.03; the p value of AAV 6×10¹¹+ERT vs NR is: 0.98; post-natal day 210: the ANOVA p value is 1.84e⁻¹³; the p value of NR vs AF is: 1.72e⁻¹²; the p value of ERT vs AF is: 0.05; the p value of AAV 2×10¹¹ vs AF is: 0.07; the p value of AAV 2×10¹¹+ERT vs AF is: 1.03e⁻⁴; the p value of AAV 6×10¹¹ vs AF is: 1.00e⁻³; the p value of AAV 6×10¹¹+ERT vs AF is: 5.73e⁷; the p value of ERT vs NR is: 1.01e⁻⁶; the p value of AAV 2×10¹¹ vs NR is: 5.50e⁻⁸; the p value of AAV 2×10¹¹+ERT vs NR is: 4.00e⁻³; the p value of AAV 6×10¹¹ vs NR is: 2.00e⁻³; the p value of AAV 6×10¹¹+ERT vs NR is: 0.99.

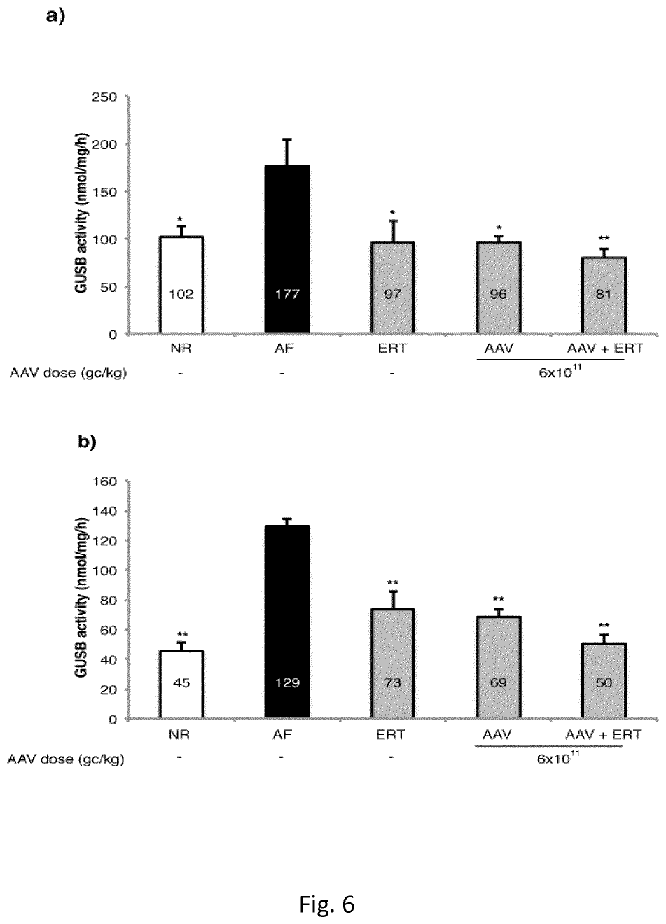

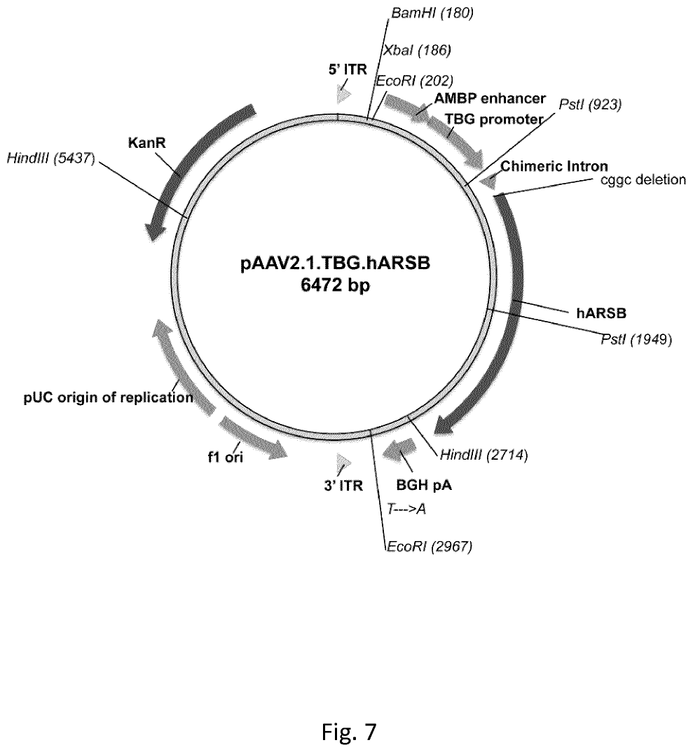

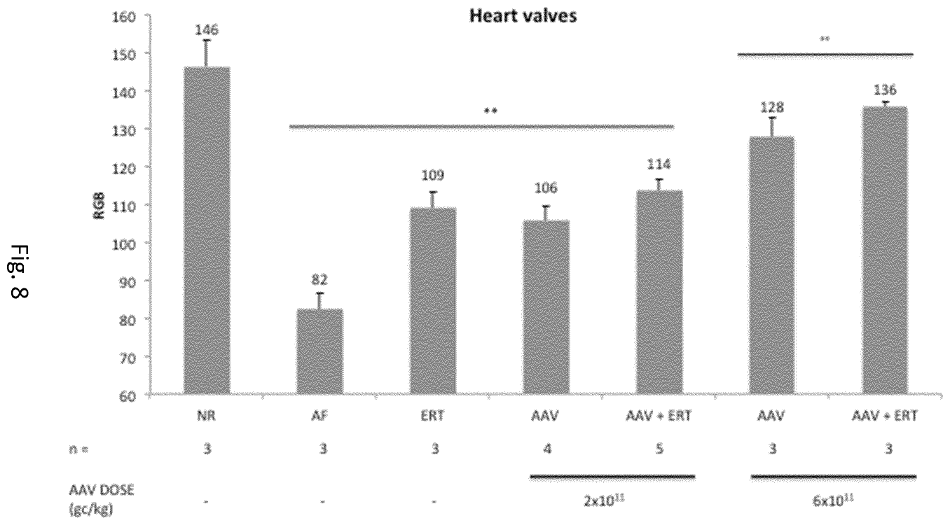

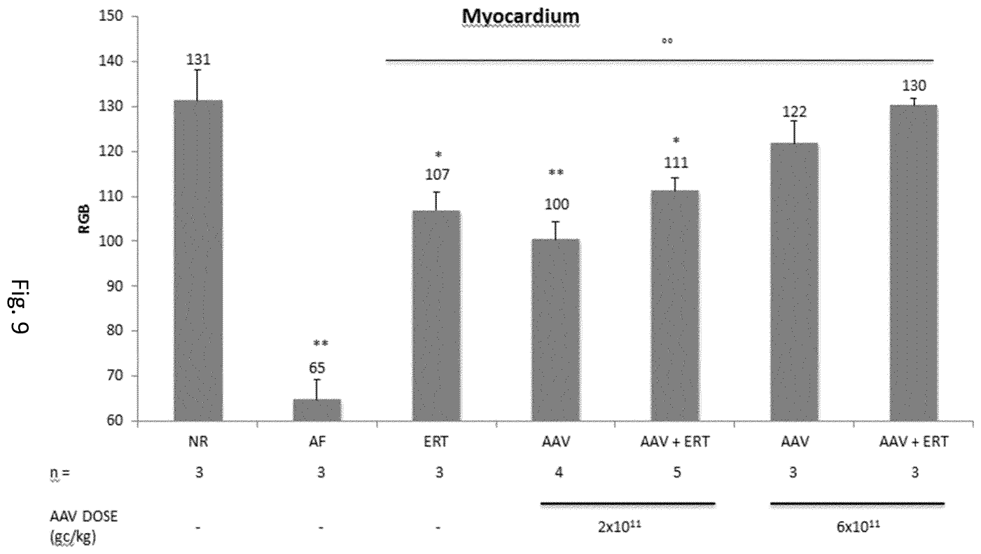

FIG. 6. Liver β-glucuronidase activity (a): The ANOVA p value is 8.88e⁻³; the p value of NR vs AF is 0.05; the p value of ERT vs AF is 0.03; the p value of AAV 6×10¹¹ vs AF is 0.03; the p value of AAV 6×10¹¹+ERT vs AF is 0.08; the p value of ERT vs NR is 0.99; the p value of AAV 6×10¹¹ vs NR is 0.99; the p value of AAV 6×10¹¹+ERT vs NR is 0.90.

Kidney β-glucuronidase activity (b): The ANOVA p value is 7.16e⁻⁷; the p value of NR vs AF is 1.00e⁻⁶; the p value of ERT vs AF is 2.50e⁻⁴; the p value of AAV 6×10¹¹ vs AF is 8.99e⁻⁵; the p value of AAV 6×10¹¹+ERT vs AF is 2.30e⁻⁶; the p value of ERT vs NR is 0.09; the p value of AAV 6×10¹¹ vs NR is 0.21; the p value of AAV 6×10¹¹+ERT vs NR is 0.99.

### EXAMPLES

### Example 1: Increased Serum ARSB Levels in MPS VI Transgenic Mice Treated with Combined Monthly ERT and Gene Therapy

MPS VI mice received at postnatal day 30 (p30) a single intravenous (i.v) administration of either 2×10¹¹ or 6×10¹¹ gc/kg of AAV2/8.TBG.hARSB, which encodes human ARSB (hARSB) under the control of the liver-specific thyroxine-binding globulin (TBG) promoter, and/or monthly i.v injections of 1 mg/kg rhARSB (Naglazyme, BioMarin Europe, London, UK), which is the dose currently used in MPS VI patients management (canonical ERT schedule)^{6, 7, 49-52}. As control, MPS VI mice were either left untreated or received a combination of monthly administrations of ERT and a single injection of the control AAV2/8.TBG.eGFP vector, which encodes the enhanced green fluorescence protein (eGFP) under the control of the TBG promoter. Serum ARSB was undetectable in affected control (AF) mice (Table 1).

TABLE 1
Liver vector genome copies, serum and peripheral tissue ARSB and GAGs in
MPS VI mice receiving low doses of gene therapy and/or monthly ERT.

| | Serum | | Liver | | Kidney | | Spleen | |
|---|---|---|---|---|---|---|---|---|
| | ARSB | | ARSB | | ARSB | | ARSB | |
| Groups | levels | gc | activity | GAGs | activity | GAGs | activity | GAGs |
| NR | 11825 ± 334** | — | 96.7 ± 7.9** | 4.5 ± 0.4** | 148.0 ± 12.0** | 7.4 ± 0.3** | 61.2 ± 4.6** | 3.0 ± 0.3** |
| AF | 0 | — | 0 | 57.0 ± 6.2 | 0 | 46.9 ± 7.8 | 0 | 49.5 ± 5.4 |
| ERT | 0 | — | 2.4 ± 0.5 | 7.3 ± 2.3** | 0.6 ± 0.3 | 15.1 ± 3.5** | 2.7 ± 1.2 | 9.2 ± 2.5** |
| AAV 2 × | 376 ± 67 | 0.015 ± 0.003 | 4.5 ± 1.2* | 8.6 ± 3.3** | 1.0 ± 0.3** | 18.9 ± 4.6** | 4.0 ± 1.0** | 14.2 ± 4.9** |
| 10¹¹ | | | | | | | | |
| AAV 2 × | 726 ± 82 | 0.034 ± 0.009 | 6.0 ± 1.6** | 5.1 ± 0.5** | 1.3 ± 0.2** | 7.3 ± 0.4** | 5.4 ± 0.5** | 6.0 ± 0.9** |
| 10¹¹ + ERT | | | | | | | | |
| AAV 6 × | 2035 ± 98* | 0.131 ± 0.038 | 11.1 ± 1.5** | 3.4 ± 0.5** | 1.4 ± 0.2** | 7.3 ± 1.0** | 5.4 ± 0.5** | 5.4 ± 0.7** |
| 10¹¹ | | | | | | | | |
| AAV 6 × | 2500 ± 84** | 0.105 ± 0.019 | 11.8 ± 0.6** | 3.3 ± 0.4** | 1.1 ± 0.1** | 6.0 ± 0.6** | 6.3 ± 1.1** | 5.1 ± 0.2** |
| 10¹¹ + ERT | | | | | | | | |

Abbreviations: AAV, adeno-associated viral vector; AF, affected MPS VI untreated mice; ARSB, arylsulfatase B; GAGs, glycosaminoglycans; ERT, enzyme replacement therapy; gc, genome copies; NR, normal untreated mice; n.a, not applicable. Dashed lines refer to values below the detection limit of the assay. Each serum ARSB value is the mean of all the time points measured in that group over time and is expressed as pg/ml. Measurements in tissues were done at the time of sacrifice (180 or 210 days of age). ARSB activity in tissues is expressed as nmol/mg protein/hour; GAG levels in tissues are expressed as μg GAG/mg protein; genome copies in livers are expressed as genome copies/molecule of diploid genome. The AAV vector dose used (gc/kg) is reported per each group. The number of animals within each treated group is: ERT, n=5 except for serum ARSB (n=4); AAV 2×10¹¹, n=6 except for serum ARSB (n=5); AAV 2×10¹¹+ERT, n=6 except for serum ARSB (n=7); AAV 6×10¹¹, n=5 except for serum ARSB n=4; AAV 6×10¹¹+ERT, n=5. The number of animals in the NR group is: 16-24 for serum ARSB [23 at post-natal day 30 (p30) and p150, 24 at p60, p90 and p120, 22 at p180 and 16 at p210];-23 for ARSB activity and GAGs levels in tissues. The number of animals in the AF group is: 9, except for serum ARSB (3-6). Genome copies in liver were analyzed in 3 un-injected (2NR and 1 ERT) mice as control. Values are represented as mean±SE. Statistical comparisons were made using the one-way ANOVA and the Tukey post hoc test. The p value vs. AF is: * <0.05 and ** <0.01. The exact p values obtained are indicated in the Material and Methods section.

MPS VI mice receiving either 2×10¹¹ or 6×10¹¹ gc/kg of AAV2/8.TBG.hARSB vector, with or without monthly ERT, showed a dose-dependent increase of serum ARSB levels, with a tendency to decrease at the end of the study (Table 1 and FIG. 1) and that corresponded to about 5% and 19% of normal (NR) levels, respectively. No significant differences in serum ARSB levels were observed in mice treated with combined gene therapy and monthly ERT compared to mice receiving only gene therapy (Table 1 and FIG. 1). However, this is expected based on the undetectable levels of serum ARSB in animals that received ERT administrations (FIG. 1), which is in accordance with the peak-and-drop serum kinetics of rhARSB infusions^{5, 6, 15}.

To confirm long-term transgene expression the inventors measured ARSB enzyme activity and AAV vector genome copies (gc) in the livers from treated and control mice at the end of the study, i.e 180 or 210 days of age (Table 1). Persistence of liver transduction was confirmed by the presence of detectable AAV vector gc in mice receiving gene therapy.

### Example 2: Increased Urinary GAG Reduction in Mice Receiving Gene Therapy in Combination with ERT

Reduction of urinary GAGs is a sensitive and reliable biomarker of lysosomal storage clearance and therapeutic efficacy in LSDs^{7, 49-52}.

Urinary GAGs were measured monthly in MPS VI-treated mice as well as in age-matched NR and AF controls, from p60, i.e. one month after the start of treatment. Urinary GAG levels measured at each time point were averaged for each group and the resulting value was reported as a percentage (%) of age-matched AF controls (FIG. 2 and FIG. 3).

Overall, urinary GAGs significantly decreased compared to AF controls in all groups of treatment (FIG. 2). GAGs reduction in urine was observed starting from one month following the beginning of treatment and was stably maintained up to the end of the study for all groups (FIG. 3). Specifically, only a slight reduction was observed in mice treated with monthly ERT, while a significant dose-dependent response was found in mice receiving gene therapy either alone (p value AAV 2×10¹¹ vs AAV 6×10¹¹: <<0.01) or in combination with ERT (p value AAV 2×10¹¹+ERT vs AAV 6×10¹¹+ERT: <<0.01).

More importantly, urinary GAGs decreased more in mice receiving the combined therapy than in those receiving the corresponding single treatments. Indeed, urinary GAGs were significantly lower (61% of AF) in mice treated with both 2×10¹¹ gc/kg of AAV and ERT than in mice treated with either monthly ERT (82% of AF, p value: <<0.01) or 2×10¹¹ gc/kg of AAV (73% of AF, p value: <<0.01). Likewise, a greater reduction of urinary GAGs was observed in mice receiving both 6×10¹¹ gc/kg of AAV and ERT (41% of AF) compared to mice treated with either ERT (p value: <<0.01) or gene therapy at the same dose (53% of AF, p value: <<0.01) (FIG. 2).

### Example 3: Amelioration of Biochemical, Visceral and Cardiac Abnormalities in MPS VI Transgenic Mice Treated with Combined Monthly ERT and Gene Therapy

ARSB activity and GAG levels were measured in the liver, kidney, and spleen of MPS VI-treated and control mice (Table 1). ARSB activity was undetectable in tissues of AF controls. MPS VI mice receiving ERT (with or without gene therapy) were sacrificed one month after the last injection of rhARSB to measure the residual tissue enzymatic activity. Although ARSB activity was almost undetectable in the serum of mice treated with ERT alone, the inventors found ARSB activity in tissues up to 1 month after injection (Table 1), although at levels lower than those previously measured in mice receiving weekly ERT¹⁵.

Increased ARSB activity was observed in the liver of all treated mice; detectable activity was variably observed in the spleen and kidney of treated mice, although at levels lower than those measured in the liver (Table 1). Specifically, a statistically significant increase in ARSB activity compared to AF was observed in all treated groups but the one that received monthly ERT.

Beta-glucuronidase (GUSB) activity has been found to be secondarily increased in tissues from MPS VI cats as result of ARSB deficiency⁵³. As further therapeutic endpoint, GUSB activity was thus evaluated in tissues of MPS VI mice treated with either the combination of 6×10¹¹ gc/kg of AAV and ERT or single therapies, and in NR and AF controls. GUSB activity was significantly increased in liver and kidney but not in spleen of MPS VI mice compared to NR (FIGS. 6a,b). While GUSB activity was completely normalized in liver regardless of the treatment, only mice receiving the combined therapy showed normalized levels of GUSB activity in kidney, although none of the groups of treatment was statistically different than NR controls (FIGS. 6a,b).

More importantly, a statistically significant reduction of GAGs storage was observed in these organs, regardless of treatment and ARSB levels (Table 1), similarly to what was observed in mice administered with weekly ERT¹⁵. This supports previous data indicating that low enzymatic levels are sufficient to improve the MPS VI visceral phenotype¹³⁻¹⁵. Specifically, tissue GAGs in all groups were not statistically different than in normal controls, except for the spleen and kidney of mice receiving 2×10¹¹ gc/kg (p value<0.05). In particular, GAGs were completely normalized in the visceral organs of all mice receiving 2×10¹¹ gc/kg and ERT or gene therapy at the dose of 6×10¹¹ gc/kg with ERT. Alcian blue staining of tissues confirmed the reduction of lysosomal GAG storage in the liver, kidney and spleen of MPS VI-treated mice (FIG. 4).

Cardiomyopathy and heart valve involvement are serious clinical complications of MPS VI that often negatively affect its prognosis⁵⁴. The MPS VI mouse mimics the human MPS VI cardiac phenotype^{15, 55}.

The inventors performed Alcian blue staining on heart histological sections from treated and control mice (FIG. 5) and found a marked reduction of GAG levels in the myocardium of MPS VI mice (FIGS. 5 and 9), with the exception of those receiving either ERT or 2×10¹¹ gc/kg of AAV, where only a slight reduction was observed. The Alcian blue staining quantification in heart valves (FIG. 8) and myocardium (FIG. 9) shows consistent reduction in mice treated with AAV in combination with ERT, where GAG storage was comparable to either NR controls. For each treatment group, similar RGB values were observed in both myocardium and heart valves despite the different extent of blue areas in these tissues, because the quantification was performed by selecting blue areas which are homogenously present in heart valves while interspersed with non-blue fibers in myocardium.

In the present invention, the inventors show that therapeutic efficacy can be obtained by combining gene therapy with rarified ERT. In particular, this was demonstrated by a great reduction of both urinary GAGs and storage in myocardium and heart valves observed in mice receiving combined monthly ERT and gene therapy. These levels of correction were similar to normal controls.

The inventors demonstrate that gene therapy may be regarded as a means to decrease the frequency of ERT infusions. While gene therapy could provide baseline enzyme levels to taper GAG levels, the high intracellular levels of therapeutic enzyme achieved with ERT can be used only occasionally to help clear tissues from any GAGs storage in excess. The use of a rarified ERT schedule should lead to several important advantages, including reduction of both allergic reaction associated with the frequent infusion of recombinant enzyme and the costs of ERT, that range between euro 150,000 and euro 450,000 per patient/year⁸ (depending on the patient weight) in the case of MPS VI. The high costs may limit the access to the therapy to patients living in less developed countries and where therapies are not supported by the public health system^{4, 8}. This scenario may change if a single administration of low dose gene therapy allows rarifying the ERT schedule.

An additional potential advantage of combining gene and protein delivery is that liver-directed gene therapy has been demonstrated to either prevent the generation of humoral immunity to the transgene product in several models of LSDs⁵⁹ or to eradicate it, if already present⁶⁹⁻⁶². Notably, immune-modulatory gene therapy with a sub-therapeutic dose of vector was shown to enhance the efficacy of ERT in murine Pompe disease by preventing the generation of humoral immunity to recombinant alfa-glucosidase^{20, 63}. Therefore, gene therapy may also positively impact on ERT therapeutic efficacy and safety by avoiding the generation of inhibitors to therapeutic proteins, which is a limit to the successful treatment of several inherited diseases. Last but not least, this study helps managing patients with LSDs for which ERT is available and who are enrolled in gene therapy clinical trials. The inventors are indeed developing a phase I/II study to test the efficacy of gene therapy for MPS VI (http://meusix.tigem.it). If efficacy is observed that is inferior to that observed during ERT, these patients who have received gene therapy could be put on a rarified rather than on the canonical highly frequent ERT schedule. In this study the inventors show in a mouse model the therapeutic efficacy of a novel combinatorial gene therapy/ERT approach for MPS VI, and potentially other LSDs. By taking advantage of the different pharmacokinetics and dynamics of either approach, this combination has the potential to reduce the risks and costs associated with gene therapy and ERT, respectively.

### REFERENCES

1. Neufeld, E F (1991). Lysosomal storage diseases. Annu Rev Biochem 60: 257-280.
2. Sands, M S, and Davidson, B L (2006). Molecular therapy: the journal of the American Society of Gene Therapy 13: 839-849.
3. Desnick, R J, and Schuchman, E H (2012). Annu Rev Genomics Hum Genet 13: 307-335.
4. Wyatt, K, et al. (2012). Health Technol Assess 16: 1-543.
5. Crawley, A C, et al. (1996). The Journal of clinical investigation 97: 1864-1873.
6. Harmatz, P, et al., (2005). Acta Paediatr Suppl 94: 61-68; discussion 57.
7. Harmatz, P, et al. (2008). Molecular genetics and metabolism 94: 469-475.
8. Schlander, M, and Beck, M (2009). Curr Med Res Opin 25: 1285-1293.
9. Mingozzi, F, and High, K A (2011). Nat Rev Genet 12: 341-355.
10. Biffi, A (2015). Hum Mol Genet.
11. Byrne, B J, et al., (2012). Human gene therapy 23: 808-815.
12. Brunetti-Pierri, N, and Auricchio, A (2010). In: Scriver, R (ed). The Metabolic and Molecular Bases of Inherited Diseases. McGraw Hill: New York.
13. Cotugno, G, et al. (2011). Molecular therapy: the journal of the American Society of Gene Therapy 19: 461-469.
14. Cotugno, G, et al. (2010). Human gene therapy 21: 555-569.
15. Ferla, R, et al., (2014). Human gene therapy 25: 609-618.
16. Ferla, R, et al. (2013). Human gene therapy 24: 163-169.
17. Hartung, S D, et al. (2004). Molecular therapy: the journal of the American Society of Gene Therapy 9: 866-875.
18. Sorrentino, N C, et al. (2013). EMBO Mol Med 5: 675-690.
19. Tessitore, A, et al. (2008). Molecular therapy: the journal of the American Society of Gene Therapy 16: 30-37.
20. Sun, B, et al. (2005). Molecular therapy: the journal of the American Society of Gene Therapy 11: 57-65.
21. Wolf, D A, et al. (2011). Neurobiol Dis 43: 123-133.
22. Ziegler, R J, et al. (2007). Molecular therapy: the journal of the American Society of Gene Therapy 15: 492-500.
23. Daly, T M, Vogler, C, Levy, B, Haskins, M E, and Sands, M S (1999). Proceedings of the National Academy of Sciences of the United States of America 96: 2296-2300.
24. Skorupa, A F, et al. (1999). Exp Neurol 160: 17-27.
25. Nathwani, A C, Rosales, C, McIntosh, J, Rastegarlari, G, Nathwani, D, Raj, D, et al. (2011). Molecular therapy: the journal of the American Society of Gene Therapy 19: 876-885.
26. Nathwani, A C, et al. (2014). The New England journal of medicine 371: 1994-2004.
27. Nathwani, A C, et al. (2011). The New England journal of medicine 365: 2357-2365.
28. Neufeld, E, and Muenzer, J (2010). The mucopolysaccharidoses. In: Scriver, R (ed). The Online Metabolic and Molecular Bases of Inherited Disease. McGraw Hill: New YorK. pp 1-73.
29. Chandler, R J, et al. (2015). The Journal of clinical investigation 125: 870-880.
30. Donsante, A, et al. (2007). Science 317: 477.
31. Nault, J C, et al. (2015). Nature genetics 47: 1187-1193.
32. Bell, P, et al. (2005). Molecular therapy: the journal of the American Society of Gene Therapy 12: 299-306.
33. Li, H, et al. (2011). Blood 117: 3311-3319.
34. Hawkins-Salsbury, J A, Reddy, A S, and Sands, M S (2011). Hum Mol Genet 20: R54-60.
35. Sands, M S, et al. (1997). The Journal of clinical investigation 99: 1596-1605.
36. Akiyama, K, et al. (2014). Molecular genetics and metabolism 111: 139-146.
37. Porto, C, et al. (2009). Molecular therapy: the journal of the American Society of Gene Therapy 17: 964-971.
38. Porto, C., et al. (2012). J Inherit Metab Dis 35: 513-520.
39. Bijarnia, S, et al. (2009). J Paediatr Child Health 45: 469-472.
40. Sillence, D, Waters, K, Donaldson, S, Shaw, P J, and Ellaway, C (2012). JIMD reports 2: 103-106.
41. Lin, D, et al., (2007). Molecular therapy: the journal of the American Society of Gene

Therapy 15: 44-52.
42. Macauley, S L, et al. (2012). Annals of neurology 71: 797-804.
43. Reddy, A S, et al. (2011). J Neurosci 31: 9945-9957.
44. Hawkins-Salsbury, J A, et al. (2015). J Neurosci 35: 6495-6505.
45. Macauley, S L, et al. (2014). J Neurosci 34: 13077-13082.
46. Eliyahu, E, Wolfson, T, Ge, Y, Jepsen, K J, Schuchman, E H, and Simonaro, C M (2011). PLoS One 6: e22447.
47. Spampanato, C, et al. (2011). Molecular therapy: the journal of the American Society of Gene Therapy 19: 860-869.
48. Heldermon, C D, et al. (2010). Molecular therapy: the journal of the American Society of Gene Therapy 18: 873-880.
49. Harmatz, P, et al. (2006). The Journal of pediatrics 148: 533-539.
50. Harmatz, P, et al. (2005). Pediatrics 115: e681-689.
51. Harmatz, P, et al. (2004). The Journal of pediatrics 144: 574-580.
52. Giugliani, R, et al. (2014). American journal of medical genetics Part A.
53. Ponder, K P, et al. (2012). Molecular therapy: the journal of the American Society of Gene Therapy 20: 898-907.
54. Braunlin, E A, et al. (2011). J Inherit Metab Dis 34: 1183-1197.
55. Strauch, O F, et al., (2003). Pediatr Res 54: 701-708.
56. Cotugno, G, et al., (2012). PLoS One 7: e33286.
57. Wang, L, et al., (2012). Human gene therapy 23: 533-539.
58. Auclair, D, et al., (2003). Molecular genetics and metabolism 78: 163-174.
59. LoDuca, P A, Hoffman, B E, and Herzog, R W (2009). Curr Gene Ther 9: 104-114.
60. Markusic, D M, et al. (2013). EMBO Mol Med 5: 1698-1709.
61. Annoni, A, et al. (2013). EMBO Mol Med 5: 1684-1697.
62. Crudele, J M, et al. (2015). Blood 125: 1553-1561.
63. Sun, B, et al. (2010). Molecular therapy: the journal of the American Society of Gene Therapy 18: 353-360.
64. Evers, M, et al. (1996). PNAS 93: 8214-8219.
65. Brooks, D A, et al. (1995). Biochem J 307 (Pt 2): 457-463.
66. Zambrowicz, B P, et al., (1997). PNAS 94: 3789-3794.
67. Chang, P L, Rosa, N E, and Davidson, R G (1981). Anal Biochem 117: 382-389.
68. Wickes, B L, and Edman, J C (1995). Mol Microbiol 16: 1099-1109.
69. de Jong, J G, et al., (1989). Clinical chemistry 35: 1472-1477.
70. Neufeld, et al., “The mucopolysaccharidoses” The Metabolic Basis of Inherited Disease, eds. Scriver et al., New York: McGraw-Hill, 1989, p. 1565-1587.
71. Braunlin E A et al., 2011. Journal of Inherited Metabolic Disease 34: 1183-1197
72. Alliegro et al., Molecualr Therapy 2016
73. de Jong, J G, Wevers, R A, Laarakkers, C and Poorthuis, B J (1989). Clin Chem 35: 1472-1477

1. (canceled)

2. A method for the treatment of MPS VI comprising:
a) administering to a subject in need thereof a vector comprising a nucleic acid encoding an arylsulfatase B and
b) administering to said subject an arylsulfatase B enzyme replacement therapy (ERT),
wherein the ERT is administered less frequently than once a week.

3. The method of claim 2 wherein the nucleic acid encodes a wild-type arylsulfatase B.

4. The method of claim 3 wherein the wild-type arylsulfatase B comprises SEQ ID No. 2 or SEQ ID No. 4.

5. The method of claim 2 wherein the nucleic acid comprises SEQ ID No. 1.

6. The method of claim 2 wherein the nucleic acid is operably linked to a liver-specific promoter.

7. The method of claim 6 wherein the liver-specific promoter is selected from the group consisting of: thyroxine-binding globulin (TBG) promoter, alfa-1-antitripsin promoter, and albumin promoter.

8. The method of claim 7 wherein the thyroxine-binding globulin (TBG) promoter comprises SEQ ID No. 11, the alfa-1-antitripsin promoter comprises SEQ ID No. 12 and the albumin promoter comprises SEQ ID No. 13.

9. The method of claim 2 wherein the vector comprises SEQ ID No. 3.

10. The method of claim 2 wherein the vector is selected from the group consisting of: an adenoviral vector, lentiviral vector, retroviral vector, adeno associated vector (AAV) and a naked plasmid DNA vector.

11. The method of claim 2 wherein the vector is an adeno-associated viral (AAV) vector.

12. The method of claim 11 wherein the AAV vector is of serotype 8.

13. The method of claim 2 wherein the vector comprises SEQ ID No. 8.

14. The method of claim 2 wherein the dosage of the vector is of from 1×10⁹ to 2×10¹⁶ gc/kg.

15. The method of claim 2 wherein the vector is administered intravenously.

16. The method of claim 2 wherein the arylsulfatase B in the ERT comprises SEQ ID No. 2 or SEQ ID No. 4.

17. The method of claim 2 wherein the arylsulfatase B in the ERT is a recombinant arylsulfatase B.

18. The method of claim 2 wherein the arylsulfatase B in the ERT is administered at a dose range of 0.001 mg/kg to 5 mg/kg.

19. The method of claim 2 wherein the arylsulfatase B in the ERT is administered intravenously.

20. The method of claim 2, wherein the arylsulfatase B enzyme replacement therapy is administered less frequently than once every 2 weeks.

21. The method of claim 2, wherein the vector is administered at a dose ranging from 2×10¹¹ gc/kg to 2×10¹² gc/kg and the arylsulfatase B enzyme replacement therapy (ERT) is administered at a dose of 1 mg/kg and less frequently than once a week, preferably once a month.

22. The method of claim 2 wherein the vector and the arylsulfatase B enzyme replacement therapy are administered at different times.

23. The method of claim 2 wherein the vector is administered prior to the initiation of the arylsulfatase B enzyme replacement therapy.

24. The method of claim 2 wherein the vector is administered simultaneously with initiation of the arylsulfatase B enzyme replacement therapy.

25. The method of claim 2 wherein the vector is administered only once.

26. The method of claim 2 wherein the vector is administered after the initiation on the arylsulfatase B enzyme replacement therapy.
